(19) United States
(12) Patent Application Publication
(10) Pub. No.: US 2021/0052839 A1
(43) Pub. Date: Feb. 25, 2021

(54) ANTERIOR INTERFACE PRESSURE MONITORING DURING VENTILATION

(71) Applicant: Covidien LP, Mansfield, MA (US)

(72) Inventors: Kun Li, San Diego, CA (US); Gabriel Sanchez, Valley Center, CA (US); Kiran Shankaranarayana, San Diego, CA (US); Mehran Shafiei, Vista, CA (US)

(73) Assignee: Covidien LP, Mansfield, MA (US)

(21) Appl. No.: 16/992,809

(22) Filed: Aug. 13, 2020

Related U.S. Application Data
(60) Provisional application No. 62/991,746, filed on Mar. 19, 2020.
(60) Provisional application No. 62/890,648, filed on Aug. 23, 2019.

(51) Int. Cl. A61M 16/00; A61M 16/20; A61M 16/06

(52) U.S. Cl. CPC A61M 16/024; A61M 16/208; A61M 16/0666; A61M 16/06; A61M 16/0063; A61M 2205/18; A61M 2016/0027; A61M 2205/502; A61M 2230/432; A61M 2205/52; A61M 2205/70; A61M 2016/003

(57) ABSTRACT

Systems and methods for monitoring delivered interface pressure comprise measuring inhalation and exhalation data to determine a virtual delivered interface pressure by a ventilator. While air pressure delivered to a patient through NIV or HFO therapy is important to patient comfort, ventilators only directly measure inhalation and exhalation data without direct measurements of delivered interface pressure. Knowledge of the delivered interface pressure may allow clinicians to more quickly titrate inhalation flow and exhalation pressure without sacrificing patient comfort. Additionally, knowledge of the delivered interface pressure in breathing circuits with high leak rates may reduce nuisance alarms that often sound when a delivered interface pressure is unknown. Associating delivered interface pressure with adjustable inhalation and exhalation parameters may allow continued monitoring of delivered interface pressure in addition to keeping delivered interface pressure within a desired range, thus optimizing patient-ventilator interaction.

### CROSS-REFERENCE TO RELATED APPLICATIONS

This application claims the benefit of U.S. Provisional Application No. 62/890,648, filed Aug. 23, 2019, and U.S. Provisional Application No. 62/991,746, filed Mar. 19, 2020, the complete disclosures of which are hereby incorporated herein by reference in their entireties.

### INTRODUCTION

Medical ventilator systems have long been used to provide ventilatory and supplemental oxygen support to patients. These ventilators typically comprise a connection for pressurized gas (air, oxygen) that is delivered to the patient through a breathing circuit (such as a flexible conduit or tubing). As each patient may require a different ventilation strategy, modern ventilators can be customized for the particular needs of an individual patient. For example, several different ventilator modes or settings have been created to provide better ventilation for patients in different scenarios, such as mandatory ventilation modes, spontaneous ventilation modes, and assist-control ventilation modes.

More recently, non-invasive ventilation (NIV) and high flow oxygen (HFO) therapy have been used to deliver breathing gases through non-invasive interfaces such as nasal prongs, nasal interfaces, or masks, rather than through invasive endotracheal tubes inserted into the trachea. NIV and HFO therapy can be less likely than invasive mechanical ventilation to cause pulmonary complications such as lung injury. However, patients commonly experience discomfort related to air pressure, particularly during the initiation of NIV or HFO therapy. Air pressure in the nasal cavity may cause a variety of discomforts, including sinus or ear pain, burning, or coldness sensations. In addition, excessive air leaks around the nasal interface or mask may cause ventilators to deliver insufficient pressure to the patient.

It is with respect to this general technical environment that aspects of the present technology disclosed herein have been contemplated. Furthermore, although a general environment has been discussed, it should be understood that the examples described herein should not be limited to the general environment identified herein.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Aspects of the disclosure relate to providing systems and methods for monitoring anterior nares pressure during NIV or HFO therapy through a nasal interface with nasal prongs. More specifically, this disclosure describes systems and methods for calibrating a ventilator connected to an NIV or HFO breathing circuit prior to patient ventilation to monitor pressure at the anterior nares without requiring an external measurement device. The estimated nares pressure provides a “virtual pressure signal” during ventilation, which can help clinicians better manage their patients in a variety of ways, including shortening the titration time of adjusting pressure settings for NIV mode, choosing the appropriate size of the interface in order to achieve the effective pressure transmission, and reducing the occurrence of nuisance alarms with a better patient disconnect detection.

In an aspect, method for calibrating a ventilator for ventilation via a non-invasive interface is provided. The method includes displaying, on a user interface of the ventilator, a prompt to connect the non-invasive interface to the ventilator and receiving a user confirmation of connection of the non-invasive interface. The method further includes commanding a flow to the non-invasive interface and iteratively reducing the commanded flow by a stepdown increment until the commanded flow reaches a baseline. Additionally, the method includes collecting a pressure measurement and a flow measurement for each stepdown increment, determining a resistance constant for the interface based on the collected measurements, and storing the resistance constant. Based on the stored resistance constant, the method further includes determining a delivered interface pressure during ventilation, and performing at least one of the following: displaying the delivered interface pressure; controlling the ventilation based at least in part on the delivered interface pressure; or determining a disconnect or reconnect status based on the delivered interface pressure.

In an aspect, the resistance constant is a Rohrer's constant. In another aspect, each stepdown increment is 1 L/min. In yet another aspect, determining the delivered interface pressure comprises measuring or estimating an airway pressure using the resistance constant. In a further aspect, the airway pressure is estimated from an inhalation airway pressure measured by the ventilator and an exhalation airway pressure measured by the ventilator. In another aspect, the airway pressure is measured by a proximal sensor located downstream of the ventilator. In a further aspect, the method further comprises adjusting the commanded flow or a controlled pressure of an exhalation valve to target a desired delivered interface pressure. In yet another aspect, the non-invasive interface comprises a nasal cannula comprising nasal prongs, and wherein the delivered interface pressure comprises a pressure delivered across an anterior tip of the nasal prongs. In another aspect, the non-invasive interface comprises a mask, and wherein the collecting operation occurs when the mask is blocked, and further wherein the delivered interface pressure comprises a pressure delivered at the mask. In yet another aspect, the method further comprises triggering an alarm if the delivered interface pressure crosses a threshold. In a further aspect, the method operates devoid of input from sensors downstream of the ventilator.

In another aspect, a method for calibrating a mechanical ventilator is provided. The method includes displaying, on a user interface of the mechanical ventilator, a prompt to connect a non-invasive interface to a dual-limb patient circuit and receiving a user confirmation of connection of the non-invasive interface to the dual-limb patient circuit. Additionally, the method includes commanding a flow to the non-invasive interface via the dual-limb patient circuit. The method further includes iteratively reducing the commanded flow by a stepdown increment until the flow reaches a baseline and collecting a pressure measurement and a flow measurement for each stepdown increment. Based on the collected pressure and flow measurements, determining a set of resistance constants for the non-invasive interface and the dual-limb patient circuit. Based on the set of resistance constants, the method includes determining a delivered interface pressure. The method further includes detecting a reconnection of the non-invasive interface to a patient, automatically resuming ventilation, and displaying, on the user interface, the delivered interface pressure during ventilation.

In an example, the method further includes displaying, on the user interface, an option to calibrate one of nasal prongs or a mask, wherein the non-invasive interface corresponds to one of the nasal prongs or the mask, and receiving a user selection of one of the nasal prongs or the mask. In another example, the method further includes displaying, on the user interface, a third prompt to remove at least one of a carbon dioxide (CO₂) sensor or a proximal flow (Prox) sensor from the dual-limb patient circuit. In yet another example, the reconnection of the non-invasive interface to the patient is determined based on the delivered interface pressure. In another example, the method further includes displaying, on a user interface of the mechanical ventilator, a prompt to disconnect the non-invasive interface from the patient, and receiving a user confirmation of disconnection of the non-invasive interface from the patient. In yet another example, the operation of detecting a reconnection of the non-invasive interface to the patient further comprises oscillating between a baseline flow and a pulse flow.

In yet another example, a method for controlling a ventilator is provided. The method includes pausing ventilation to calibrate the ventilator with a nasal prong interface comprising nasal prongs and controlling a flow valve of the ventilator to execute a stepdown flow calibration with the nasal prong interface open to ambient air. Additionally, the method further includes updating a set of resistance values based on a plurality of measurements from the stepdown flow calibration and resuming ventilation, comprising delivering breathing gases to the nasal prong interface. The method further includes determining a delivered interface pressure from the updated set of resistance values, wherein the delivered interface pressure comprises a pressure delivered at an anterior end of the nasal prongs. Additionally, the method includes displaying the delivered interface pressure on a display screen of the ventilator.

In another example, the method further includes receiving a target interface pressure and controlling the flow valve to drive the delivered interface pressure toward the target interface pressure. In yet another example, the method further includes determining a disconnect of the nasal prong interface based on the delivered interface pressure and a threshold pressure.

It is to be understood that both the foregoing general description and the following Detailed Description are exemplary and explanatory and are intended to provide further explanation of the disclosure as claimed.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is an illustration of a ventilator capable of providing interface pressure monitoring during ventilation.

FIG. 2 is block diagram that illustrates an example of a ventilatory system coupled to a non-invasive interface.

FIG. 3A is an illustration of a schematic diagram of a breathing circuit and non-invasive interface at steady state, in the form of an electrical circuit diagram.

FIG. 3B is an illustration of a schematic diagram of a breathing circuit and non-invasive interface during dynamic ventilation, in the form of an electrical circuit diagram.

FIG. 4A is a graphical illustration of parameters measured and collected during a stepdown flow calibration of a non-invasive, resistive interface.

FIG. 4B is a graphical illustration of parameters measured and collected during a stepdown flow calibration of a non-invasive, non-resistive interface.

FIG. 4C is a graphical illustration of a parameter measured during a disconnect phase to detect a patient reconnect to a non-invasive interface.

FIGS. 5A-D are flowcharts illustrating a method for calibrating a ventilator for interface pressure monitoring.

FIGS. 6A-E are representations of a graphical user interface for anterior interface calibration and pressure monitoring, according to examples.

FIG. 7 is a block-diagram illustrating an example of a ventilatory system for anterior interface pressure monitoring during ventilation.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

Aspects of the disclosure relate to providing systems and methods for monitoring patient interface pressure during ventilation (e.g., NIV, HFO therapy, or other ventilation) from a medical ventilator through a patient interface (e.g., nasal prongs, mask, or other interfaces). In a clinical setting, “anterior” refers to the direction toward the patient's back (if lying supine, this is downward toward the floor), and “posterior” refers to the direction toward the patient's chest (if lying supine, this is upward toward the ceiling). The “anterior nares pressure” refers to the pressure of breathing gases exiting the anterior tip of the nasal prongs or cannula, inside the patient's nose. This anterior nares pressure is the pressure actually delivered across the interface into the patient's airway, and is thus the pressure that is clinically relevant to adequately ventilate the patient. A “mask pressure” (Pₘₐₛₖ) refers to the pressure of breathing gases existing in a mask (covering the nose, mouth, or both) and delivered across the interface into the patient's airway. The anterior nares pressure or the mask pressure may also be referred to as the anterior interface pressure or the delivered interface pressure. Too little pressure delivered across the interface into the patient's airway can cause under-ventilation (insufficient oxygenation or CO₂ removal), while too much pressure can cause lung injury. Due to the pressure absorbed along the patient circuit (the flexible breathing tube) and the nasal or mask interface itself, the delivered interface pressure is lower than the pressure applied upstream by the mechanical ventilator. In particular, when small nasal prongs are used for very small patients (such as pre-term infants), the pressure across the nasal prongs can drop significantly from the pressure applied by the ventilator. Other non-invasive interfaces may also cause a deviation in the pressure delivered to the patient from the pressure applied by the ventilator (e.g., a mask pressure, Pₘₐₛₖ, existing at the mask may be different from the pressure applied by the ventilator). The deviation in delivered interface pressure may also result in inaccurate patient disconnect and reconnect detection. Accordingly, the present disclosure relates to systems and methods for estimating and monitoring the delivered interface pressure.

Although the techniques introduced above and discussed in detail below may be implemented for a variety of medical devices, the present disclosure will discuss the implementation of these techniques in the context of delivered interface pressure monitoring during NIV or HFO therapy. A person of ordinary skill in the art will understand that the application of the technology to NIV and HFO therapy could be adapted to other ventilation types—including anterior or delivered pressure across an endotracheal tube during invasive mechanical ventilation—and may be used in a variety of breathing circuit setups. Moreover, although this disclosure describes determining a relationship between delivered interface pressure and inhalation flow, inhalation pressure, exhalation flow, and exhalation pressure, other ventilation parameters may be used to determine delivered interface pressure.

Medical ventilators are used to provide breathing gases to patients who are otherwise unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating flow valves function to regulate flow so that respiratory gases having a desired concentration are supplied to the patient at desired pressures and flow rates. Modern ventilators monitor, evaluate, and graphically represent a myriad of ventilatory parameters. However, delivered interface pressure in NIV or HFO therapy or other forms of ventilation is not one of those determined parameters, due in part to the difficulty and impracticality of placing a pressure or flow sensor (or other measurement device) at the anterior tip of the prongs within the patient's nose, or at the exit or tip of other patient interfaces (invasive or non-invasive).

Research has shown that the pressure transmitted across the nasal interface (from the patient circuit to the anterior nares) is lower than the pressure applied upstream by the ventilator. (A nasal interface with nasal prongs is described here as an example, but it should be understood that the interface could include other types of patient interfaces such as nasal or mouth masks.) Nasal prongs are typically much smaller in diameter than the patient circuit (the tube connecting the nasal prongs to the ventilator), and the reduction in diameter causes the flow of air to increase in speed and drop in pressure. Additionally, the transmission of pressure across the nasal prongs may be impacted by other factors including (1) leaks at the interface, and (2) the size of the interface. When the patient interface is properly sized, good pressure transmission can be achieved, but if the nasal interface is too small for the patient's nasal nares, pressure transmission is reduced. For at least these reasons, clinicians may not understand what pressure is actually being transmitted to the patient during NIV or HFO therapy with a noninvasive interface.

Furthermore, some ventilators may have difficulty distinguishing a patient disconnect occurrence (when the patient becomes disconnected from the breathing circuit) from air leaks around the interface. Air leaks around the interfaces are common during NIV and HFO therapy. Some mechanical ventilators working under NIV modes estimate leak around the interface and compare the leak to a threshold. If the estimated leak is above that threshold, the ventilator determines that the patient is no longer connected to the circuit and declares a high priority disconnect alarm. However, a large variable leak is common during NIV and HFO therapy, particularly when the patient is awake and moving. When this leak frequently crosses the alarm threshold, it will cause intermittent circuit disconnect detection and lead to frequent nuisance alarms, which is annoying to both the clinician and patient.

According to an example of the present disclosure, a system and method are provided for estimating and monitoring anterior interface pressure during non-invasive ventilation of a patient on a mechanical ventilator. A method is provided for calibrating the ventilator based on the non-invasive interface selected for the patient. The calibrated ventilator can estimate and monitor delivered interface pressure (such as anterior nares pressure or mask pressure) without the need for additional pressure or flow sensors at the interface or along the patient circuit. Accurate estimation of delivered interface pressure may improve patient care by shortening the amount of time the patient spends on mechanical ventilation and reducing the incidence of inadequate or excessive ventilation. An accurate estimation of the delivered interface pressure may also reduce nuisance alarms by providing the ventilator with an additional pressure parameter for disconnect monitoring. Additionally, the method can be applied to invasive mechanical ventilation to estimate and monitor the pressure delivered across the anterior end of the endotracheal tube inside the patient.

With these broad concepts in mind, several examples of delivered interface pressure monitoring methods and systems are discussed, below.

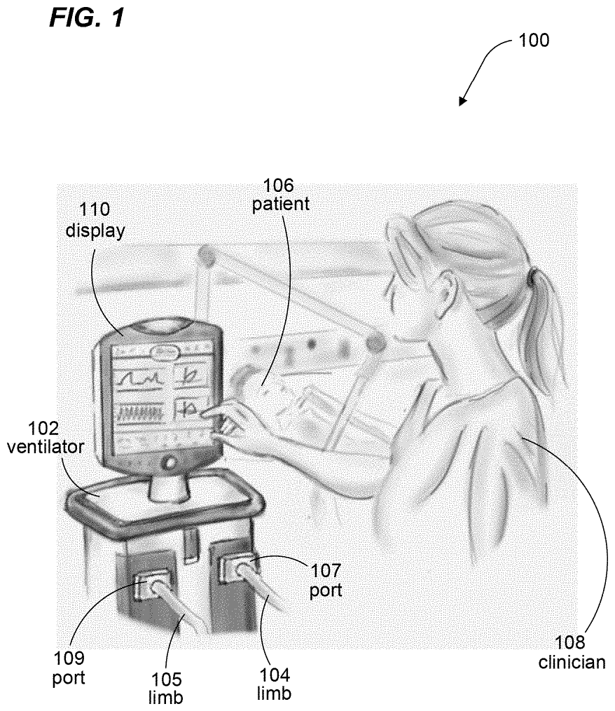

For example, FIG. 1 is a schematic diagram 100 that illustrates a ventilator 102 capable of monitoring delivered interface pressure during ventilation. As shown, the schematic diagram 100 comprises a ventilator 102, patient 106, clinician 108, and display 110. The ventilator 102 may engage one or more data collection sensors (not shown) to monitor various parameters that may be measured or calculated based on the closed system between the ventilator 102 and the patient 106. For example, the data collection sensors may collect one or more of gas flow, pressure, volume, or any other data or parameter that may be measured, calculated, or derived based on ventilation of the patient 106, measured at either or both the inhalation port 107 and exhalation port 109 of the ventilator. In an example, the ventilator 102 includes pressure and flow sensors at the inhalation port 107 that measure pressure and flow of the inhalation gases flowing into the inhalation limb 104 of a breathing circuit to the patient 106, and pressure and flow sensors at the exhalation port 109 that measure pressure and flow of the exhalation gases returning through the exhalation limb 105 of the breathing circuit to the ventilator from the patient 106. The ventilator 102 may also receive pressure and flow measurements from sensors along the breathing circuit, but these are optional, as discussed further below. This measured, collected, or calculated data may be used by the clinician 108 or ventilator 102 when determining potential adjustments or changes to settings of the ventilator 102 in order to optimize patient-ventilator interaction. In an example, the breathing circuit is connected to a non-invasive interface such as nasal prongs or a nasal, facial, or mouth mask.

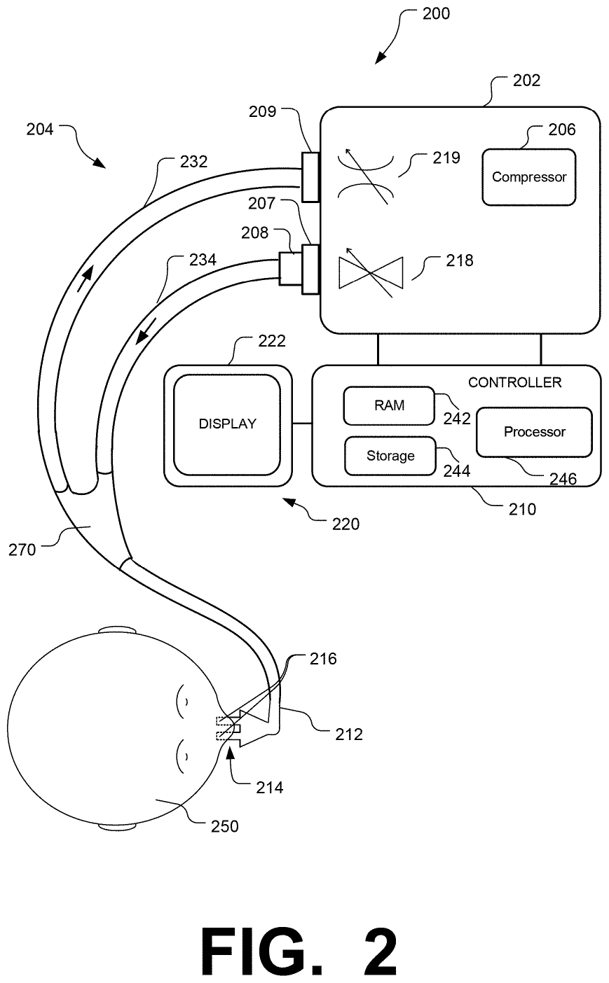

FIG. 2 is a block diagram that illustrates a ventilator 200 connected to a dual-limb breathing circuit 204 connected to a noninvasive patient interface 212, which is connected to a human patient 250. The breathing circuit 204 extends from the inhalation port 207 of the ventilator to the nasal interface 212, and from there back to the exhalation port 209 of the ventilator 200. In this example, the noninvasive nasal interface 212 includes two nasal prongs 214 sized to fit inside the patient's nostrils (the nasal nares), however, other non-invasive interfaces may be supported (e.g., mask). The prongs 214 extend the air flow path from the patient circuit 204 into the patient's nares. The air flow exits the nasal interface at the anterior tips 216 of the prongs 214. The ventilator 200 controls the flow of gases into and out of the patient circuit by controlling (adjusting, opening, or closing) an inhalation flow valve 218 and an exhalation valve 219. Additionally, a humidifier 208 may be placed along the breathing circuit 204 to humidify the inhalation gases to enhance comfort for the patient 250. Pressure and flow sensors are located at the inhalation and exhalation ports 207, 209 to measure parameters of the inhalation and exhalation flows.

Ventilator 200 includes a pneumatic system 202 (also referred to as a pressure generating system 202) for circulating breathing gases to and from patient 250 via the patient circuit 204 and non-invasive interface 212. The patient circuit 204 is a two-limb flexible tube for carrying gases to and from the patient 250. A fitting, typically referred to as a “wye-fitting” 270, connects an inhalation limb 234 and an exhalation limb 232 of the circuit, and couples the circuit to the patient interface 212.

The inhalation limb 234 is connected to the inhalation port 207 and to the inhalation flow valve 218, and the exhalation limb 232 is connected to the exhalation port 209 and the exhalation valve 219. A compressor 206 or other source(s) of pressurized gases (e.g., tanks or hoses that supply compressed air, oxygen, and/or helium) provides a gas source for ventilatory support via inhalation limb 234. The pneumatic system 202 may include a variety of other components, including mixing modules, valves, sensors, tubing, accumulators, filters, etc. Controller 210 is operatively coupled with pneumatic system 202, signal measurement and acquisition systems, and an operator interface 220 that may enable an operator to interact with the ventilator 200 (e.g., change ventilator settings, select operational modes, view monitored parameters, etc.). Controller 210 may include hardware memory 242, one or more processors 246, storage 244, and/or other components of the type commonly found in command and control computing devices. In the depicted example, operator interface 220 includes a display 222 that may be touch-sensitive and/or voice-activated, enabling the display 222 to serve both as an input and output device.

In an example, the ventilator 200 controls the breathing gases to and from the patient based on the pressure and flow sensors that are internal to the ventilator 200, and without measurements from any sensors external to the ventilator such as flow sensors along the breathing circuit 204. For example, in the example of FIG. 2, the ventilator has no direct measurement information along the circuit 204 or at the anterior tip 216 of the prongs 214. That is, the ventilator operates without input from any pressure, flow, temperature, or other sensors along the circuit 204 or nasal interface 212. Based on the sensors internal the ventilator 200, the ventilator is able to control the breathing gases (through operation of the inhalation flow valve 218 and exhalation valve 219) and estimate measured parameters along the circuit 204 and nasal interface 212.

In an example, based on its internal sensors and a calibration routine, the ventilator 200 estimates the delivered interface pressure—the pressure of breathing gases exiting the anterior tip 216 of the nasal prongs or cannula, inside the patient's nose (or in the case of a mask, the mask pressure).

In an example, the ventilator 200 estimates delivered interface pressure based on a relationship between delivered interface pressure and one or more air flow parameters measured by the ventilator in real time during ventilation. For example, the measured air flow parameter may be net flow (the difference between inhalation and exhalation flow, measured at the inhalation and exhalation ports of the ventilator), airway pressure at the wye 270, or other suitable parameters. The ventilator can estimate delivered interface pressure in real-time during ventilation of a patient, based on the measurements at the ventilator.

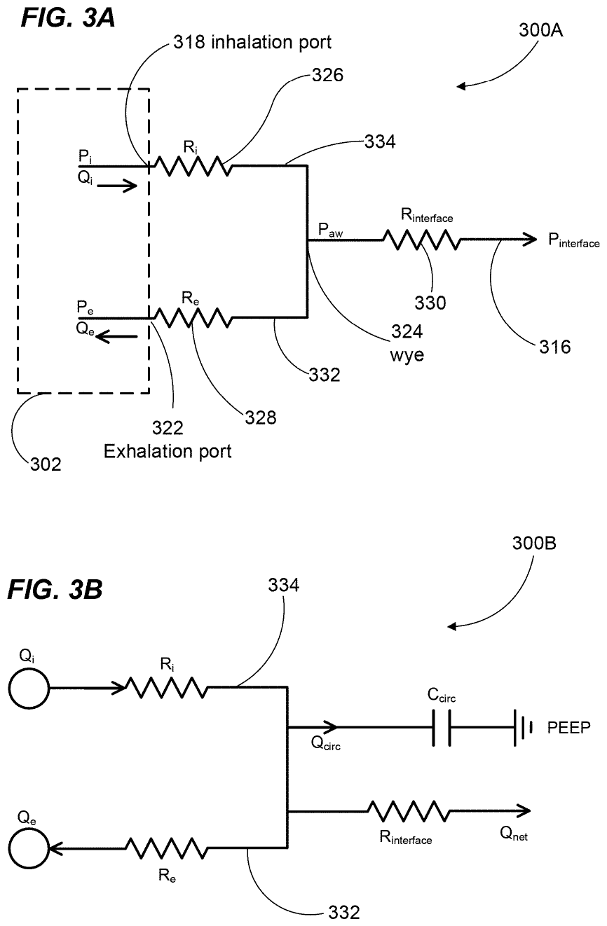

An example mathematical model relating delivered interface pressure to other ventilation parameters is depicted in FIG. 3A. FIG. 3A is a schematic diagram that illustrates the breathing circuit and non-invasive resistive interface of FIG. 2 in the form of a circuit diagram 300A. In this model, the airflow is modeled as an electrical current, and the resistance of the airflow along a path is modeled as an electrical resistor R. The circuit diagram 300A begins at the ventilator 302 where one or more sensors at an inhalation port 318 measure inhalation flow, Qᵢ, and inhalation pressure, Pᵢ, of gases passing through the inhalation flow valve. The inhalation flow then travels through an inhalation limb 334 of the breathing circuit with an inhalation resistance 326 (Rᵢ) before reaching the circuit wye 324 (the intersection of the circuit with the nasal interface). The pressure at the circuit wye 324 may be referred to as airway pressure (P_{aw}) or wye pressure (P_{wye}), and this value may be either estimated (by the ventilator, based on its internal sensors) or measured (by an external pressure or flow sensor located at the wye 324). The air flow to the anterior tip 316 is associated with interface resistance 330 (R_{interface}, such as prong resistance) and results in an interface pressure (P_{interface}), and the air flow returning through an exhalation limb 332 of the breathing circuit is associated with an exhalation resistance 328 (Rₑ). One or more sensors at the exhalation port 322 of the ventilator 302 may measure an exhalation pressure, Pₑ, and an exhalation flow, Qₑ.

Based on the electrical circuit diagram of FIG. 3A, a relationship between the delivered interface pressure (e.g., in the case of a nasal cannula, anterior nares pressure, Pₙₐᵣₑₛ) and the other air flow parameters (such as Pᵢ, Qᵢ, Pₑ, Qₑ) can be modeled. The circuit diagram 300A in FIG. 3A represents the patient circuit and, as shown, a nasal interface at steady state, when pressure and flow are relatively steady and not changing dynamically. This is the case, for example, during a calibration routine where the ventilator 302 applies a set pressure or flow at set increments, holding the pressure and flow steady in order to take measurements for calibration. During calibration, the ventilator commands a series of pressure values at different increments and measures the corresponding flow and pressure at the inhalation and exhalation ports. With these measurements, the ventilator is able to identify relationships between the commanded interface flow and the delivered interface pressure and the corresponding measured inhalation flow, Qᵢ, inhalation pressure, Pᵢ, exhalation flow, Qₑ, and exhalation pressure, Pₑ. After collecting a plurality of measurements of inhalation flow, Qᵢ, inhalation pressure, Pᵢ, exhalation flow, Qₑ, and exhalation pressure, Pₑ, the anterior nares pressure (or delivered interface pressure) may be non-linearly modeled using the following resistance-flow model equations derived from FIG. 3A:

$${R}_{i} = \frac{Δ {P}_{i}}{{Q}_{i}} = {β}_{0 , i} + {β}_{1 , i} \cdot {Q}_{i}$$

$${R}_{e} = \frac{Δ {P}_{e}}{{Q}_{e}} = {β}_{0 , e} + {β}_{1 , e} \cdot {Q}_{e}$$

$${R}_{prong} = \frac{Δ {P}_{prong}}{{Q}_{net}} = {β}_{0 , prong} + {β}_{1 prong} \cdot {Q}_{net}$$

$$Δ {P}_{i} = {P}_{i} - {P}_{aw} \\ Δ {P}_{e} = {P}_{aw} - {P}_{e} \\ Δ {P}_{prong} = {P}_{aw} - {P}_{nares} \\ {Q}_{net} = {Q}_{i} - {Q}_{e}$$

Where β_{0,i}, β_{1,i}, β_{0,e}, β_{1,e}, β_{0,prong}, and β_{1,prong}, are Rohrer's constants (which also may be referred to herein as resistance constants). Rohrer's constants come from Rohrer's equation, an empiric model for airway resistance. It is expressed as:

R=β₀+β₁×Q

where R is the resistance, Q is the volumetric flow rate, β₀ is the coefficient of laminar flow, and β₁ is a coefficient of turbulent flow.

By controlling and varying flow and pressure during calibration, Rohrer's constants may be calculated and may be stored, as described more fully below.

If the non-invasive interface is a non-resistive interface such as a mask, then the interface resistance 330 is assumed to be zero. The mask itself is assumed to be open to ambient and not present any resistance to flow. However, the mask may be attached to a small amount of tubing that changes the resistance of the breathing circuit (such as circuit 204) connected to the patient. Thus, new Rohrer's constants are calculated for the mask circuit, by comparing Rohrer's constants of the breathing circuit including the mask interface with Rohrer's constants of the breathing circuit without the mask interface (which may be obtained during a short self test, “SST,” of the ventilator during startup). The wye 324 pressure (P_{aw}) calculated for the mask circuit may be estimated as the mask pressure applied to the patient, without calculating an interface resistance 330.

FIG. 3B shows another diagram 300B, this time modeling the patient circuit and nasal interface during dynamic ventilation, when the pressure and flow applied from the ventilator are changing. In this model, the patient circuit includes inhalation flow Qᵢ and resistance Rᵢ across the inhalation limb 334, and exhalation flow Qₑ and resistance Rₑ across the exhalation limb 332. As before, the non-invasive interface is modeled with net flow Qₙₑₜ across an interface resistance R_{interface} (or R_{if}). Additionally, this model includes a branch with a circuit flow Q_{circ} and compliance C_{circ}. This branch models the flow absorbed by the patient circuit (the flexible tube of breathing circuit 204 of FIG. 2) during dynamic changes in flow and pressure applied by the ventilator. When the pressure across the patient circuit is steady and not changing, the circuit itself does not absorb or release flow, and Q_{circ} is zero or near zero. But when the ventilator is increasing or decreasing the pressure that it is applying (from the inhalation or exhalation valves), the patient circuit itself will absorb some of those changes due to its flexibility and compliance. The pressure maintained in the circuit manifests as PEEP (positive end exhalation pressure), the positive airway pressure applied back against the patient during exhalation.

FIG. 3B includes Q_{circ} and C_{circ} to account for this behavior of the patient circuit (the tube of the breathing circuit 204 from FIG. 2) during dynamic ventilation. In the diagram 300B, Q_{circ} is modeled in order to obtain an accurate estimation of Qₙₑₜ (the flow delivered to the patient across the anterior end of the interface). Q_{circ} and associated P_{circ} are applied by the ventilator but are absorbed by the circuit and do not flow into the patient's lungs. This model accounts for some of the difference between the pressure applied by the ventilator and the lower pressure actually delivered to the patient's airway.

FIGS. 3A and 3B show two models of airflow in a ventilation circuit, but it should be understood that other models may be used. Other models may include, for example, higher order or higher fidelity models based on electrical resistance theories, or lookup tables based on interpolation of values, or various types of mathematical models based on the physical law (another example is the Hagen-Poiseuille equation describing the pressure drop in a fluid flowing through a long cylindrical pipe).

After determining the Rohrer's constants to calibrate the resistance characteristics of the breathing circuit, additional calculations may be performed. When the interface is a nasal cannula (or nasal interface), two additional calculations may be performed prior to calculating the anterior nares pressure/prong pressure (i.e., delivered interface pressure for a breathing circuit with a nasal interface): (1) the airway pressure, P_{aw}, and (2) the pressure gradient across the prong.

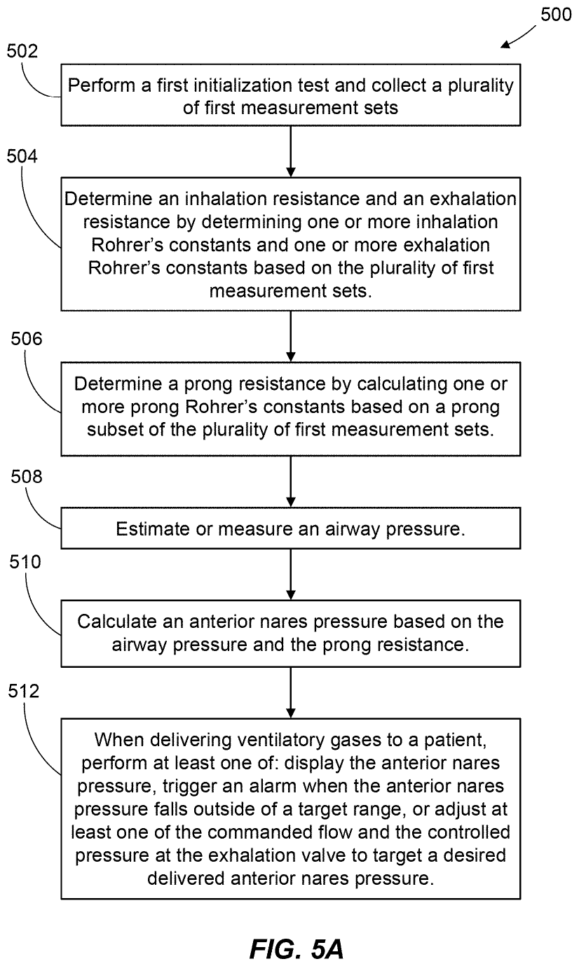

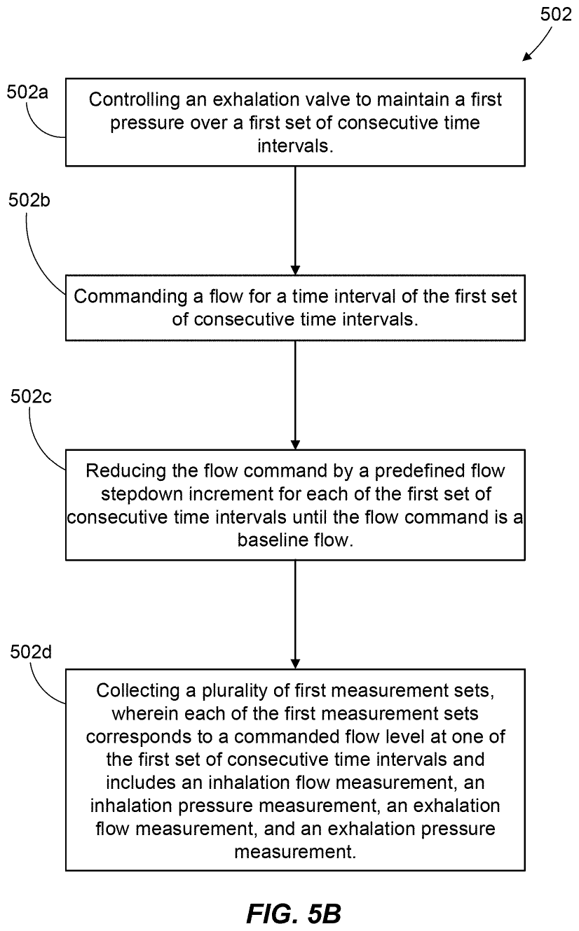

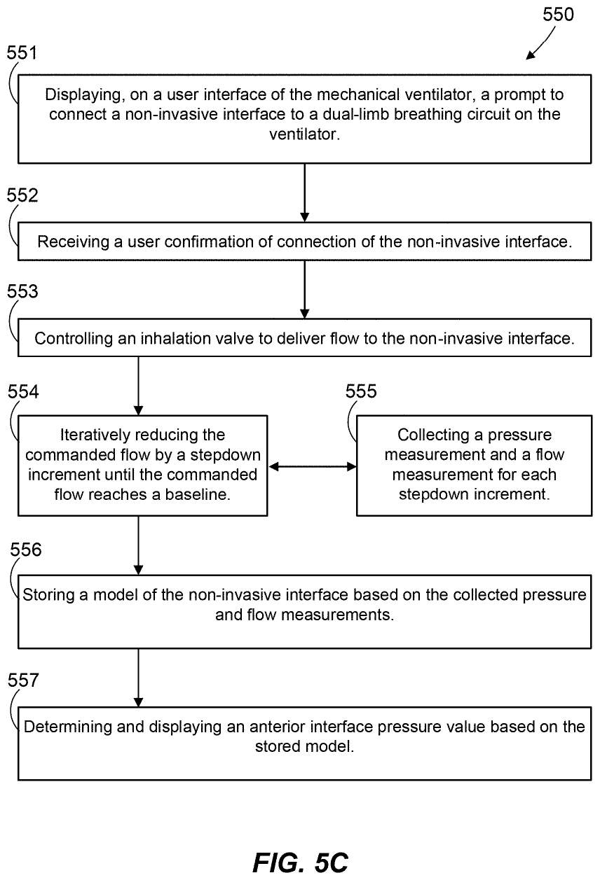

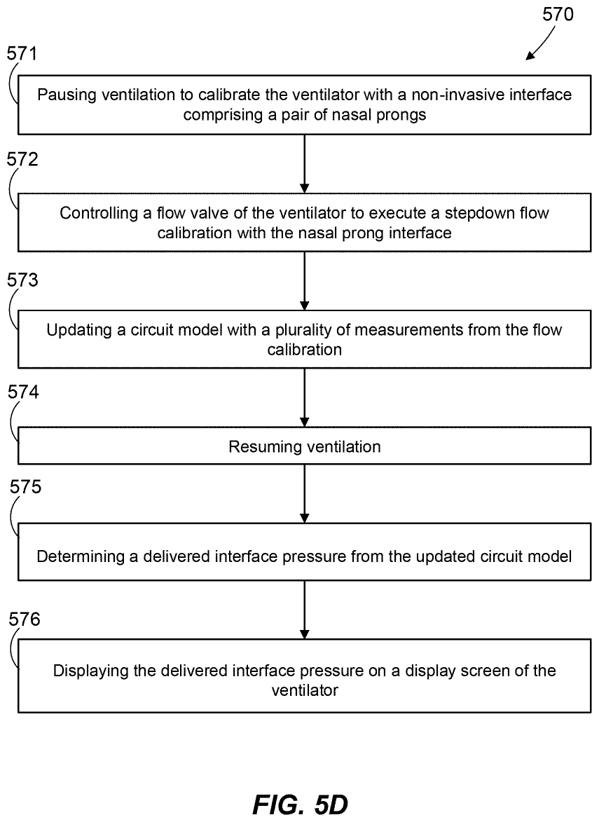

(1) The airway pressure, P_{aw}, may be estimated at the circuit wye 324 by using pressure and flow measurements at the inhalation port 318 and exhalation port 322 of the ventilator 302. As an example, two independent estimates of the airway pressure, one from the inhalation limb and one from the exhalation limb, may be averaged together in a weighted average based on relative confidence or error. This is described further below in connection with operation 508 in FIG. 5A.

(2) The pressure gradient across the prong may be calculated using the following resistance model derived from FIGS. 3A and 3B and previously-calculated Rohrer's constants (calculated during calibration of the interface), in the following equation:

ΔP_{prong}=R_{prong}·Qₙₑₜ=(β_{0,prong}+β_{1,prong}·Qₙₑₜ)·Qₙₑₜ

The delivered interface pressure (e.g., anterior nares pressure) may be calculated, using the following equation:

P_{interface}=P_{aw}−ΔP_{interface}

The Rohrer's constants may vary by breathing circuit 204 and type of interface 212. The diameter, shape, length, material, connectors, intersection angles, or layout of the breathing circuit 204 may affect the estimated value of the Rohrer's constants and the resulting anterior nares pressure.

The Rohrer's constants used above are determined during a calibration exercise that is executed by the ventilator 102 prior to ventilating the patient 106 with a selected interface. In an embodiment, a calibration exercise is executed for each interface that is used with the patient. That is, if a first interface (such as a mask) is swapped out for a second different interface (such as a differently sized mask, or a nasal cannula), then the calibration routine is re-executed to obtain Rohrer's constants for the new interface. Changing the interface without re-calibration may lead to inaccurate delivered interface pressure estimations.

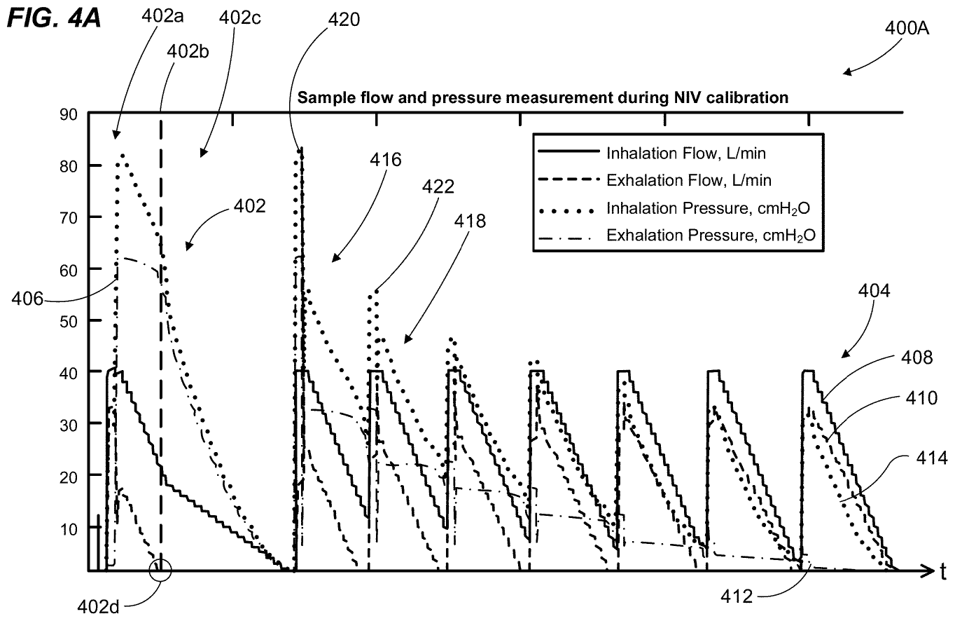

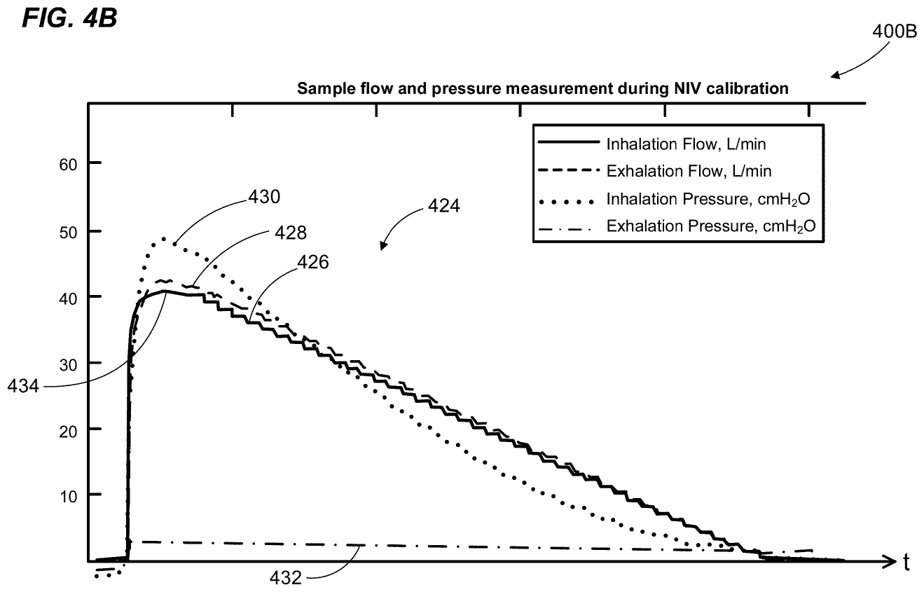

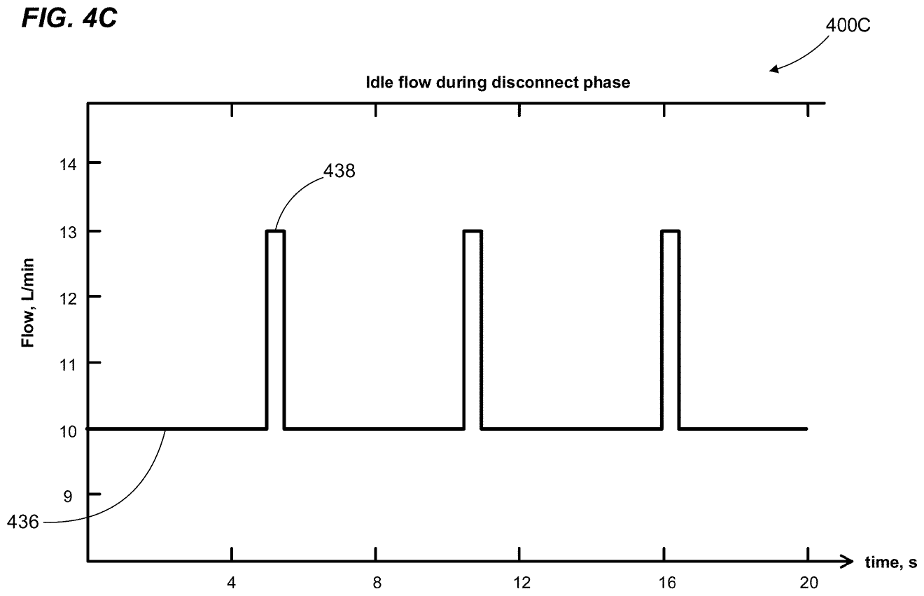

Example calibrations to collect data for the mathematical models described herein are depicted in FIG. 4A and FIG. 4B. FIG. 4 is a graphical illustration 400A of parameters measured and collected during an example calibration of a non-invasive, resistive interface with an interface resistance (e.g., interface resistance 330, such as for nasal prongs). As shown, the graphical illustration 400A shows inhalation flow 408, exhalation flow 410, inhalation pressure 414, and exhalation pressure 412 graphed as a function of time as measured by a ventilator. To apply the above calculations to derive an estimated value for the delivered interface pressure, the ventilator runs a calibration routine. The calibration routine operates a plurality of initialization tests to collect a plurality of data points. In this example, there is a pre-initialization test 402 and seven initialization tests shown, with the pre-initialization test 402 representing the first spike in values, and the seventh initialization test 404 representing the eighth spike in values. In other examples, there may be more or less than seven or eight initialization tests as shown. The duration of the calibration may be adjustable, by reducing or increasing the number of initialization tests that are performed during the calibration. More initialization tests may produce more accurate delivered interface pressure calculations but may take more time, while fewer initialization tests may result in less accurate estimates in a shorter time. Accuracy and time may be balanced to determine an ideal number of initialization tests for a clinical setting.

During calibration, the patient circuit and non-invasive interface are connected to the ventilator, but not to the patient. The anterior tip of the interface is left open to the ambient air. The pre-initialization test 402 is split into two stages: a first stage 402a and a second stage 402c. The transition from the first stage 402a to the second stage 402c of the pre-initialization test 402 is shown by the dashed transition line 402b. During the first stage 402a of the pre-initialization test 402, the ventilator commands a pressure sufficient to close the exhalation valve (referred to herein as a “closing pressure”) (e.g., closing pressure 406) and maintain the closing pressure for the entire period of the first stage 402a of the pre-initialization test 402. Additionally, during first stage 402a, the ventilator commands a flow and reduces the flow by a first preliminary flow stepdown increment over a first preliminary set of consecutive time intervals until the flow command is associated with a negligible exhalation flow 402d. For example, during the first stage 402a, the closing pressure 406 may be 60 cmH₂O and the flow command may be 40 L/min for a first time interval of 600 ms, with a first preliminary flow stepdown increment of 2 L/min, and negligible exhalation flow 402d of about 20 L/min. At each flow command (e.g., 40 L/min, 38 L/min, 36 L/min, . . . , about 20 L/min), the ventilator may record a measurement set including an inhalation flow measurement, inhalation pressure measurement, exhalation flow measurement, and exhalation pressure measurement. These measurements are compiled into a plurality of first preliminary measurement sets for the first stage 402a of the pre-initialization test 402. The dashed transition line 402b represents the time at which the ventilator measures a negligible exhalation flow 402d, and indicates a transition of the pre-initialization test 402 from the first stage 402a to the second stage 402c.

In the second stage 402c of the pre-initialization test 402, the ventilator no longer maintains the closing pressure at the exhalation valve. Here, the ventilator commands a flow and reduces the commanded flow by a second preliminary flow stepdown increment over a second preliminary set of consecutive time intervals until the flow command reaches a preliminary baseline flow. In this example, the flow command is about 20 L/min for the first time interval of one second, with a second preliminary flow stepdown increment of 1 L/min, and a preliminary baseline flow of about 1 L/min. At each flow increment (e.g., 20 L/min, 19 L/min, 18 L/min, . . . , 1 L/min), the ventilator records an inhalation flow measurement, inhalation pressure measurement, exhalation flow measurement, and exhalation pressure measurement. These measurements are compiled into a plurality of second preliminary measurement sets for the second stage 402c of the pre-initialization test 402.

After conducting the pre-initialization test 402, but prior to conducting a first initialization test 416, the ventilator may exhibit a first transient response for a time period. For example, when transitioning from the pre-initialization test 402 to the first initialization test 416, the ventilator may fluctuate in amperage to adjust the commanded pressure from the last pre-initialization test pressure to a first pressure for the first initialization test (as described, below), causing a spike 420 in the inhalation and exhalation pressures.

For a first initialization test 416, the ventilator commands a flow for a time interval of a first set of consecutive time intervals (e.g., each time interval being 600 ms), and reduces the flow by a flow stepdown increment until the flow command is a baseline flow (e.g., about 10 L/min). Throughout the first initialization test, the ventilator may command and maintain a first pressure of the exhalation valve. For example, the first pressure may be 30 cmH₂O and is maintained on the exhalation valve for the entirety of the first initialization test. In this example, the flow command is 40 L/min for a first time interval of 600 ms, with a flow stepdown increment of 2 L/min (e.g., 40 L/min, 38 L/min, 36 L/min, . . . , X L/min). X is the final flow increment, which is reached when the measured exhalation flow is recorded at a threshold value, for example, a threshold value of 0.3 L/min (which is associated with a baseline flow of X=about 10 L/min). At each flow increment, the ventilator records an inhalation flow measurement, inhalation pressure measurement, exhalation flow measurement, and exhalation pressure measurement, which are compiled into a plurality of first measurement sets for the first initialization test 416.

After conducting the first initialization test 416, but prior to conducting a second initialization test 418, the ventilator may exhibit a second transient response for a time period, similar to the first transient response. For example, when transitioning from the first initialization test to the second initialization test, the ventilator may fluctuate in amperage to adjust the commanded pressure from the first pressure of the first initialization test to a second pressure for the second initialization test (as described, below), causing the spike 422 in the inhalation and exhalation pressures.

For a second initialization test 418, the ventilator may command a second pressure at the exhalation valve. For example, the second pressure at the exhalation valve may be 25 cmH₂O. Additionally, during the second initialization test 418, the ventilator commands a flow for a first time interval, and reduces the flow command by a flow stepdown increment over the second set of consecutive time intervals until a threshold value is reached. For example, the flow may be 40 L/min for a first time interval of 600 ms with a flow stepdown increment of 2 L/min. At each flow increment (e.g., 40 L/min, 38 L/min, 36 L/min, . . . , X L/min), the ventilator records an inhalation flow measurement, inhalation pressure measurement, exhalation flow measurement, and exhalation pressure measurement, which are compiled into a plurality of second measurement sets for the second initialization test 418.

As described above, the exhalation valve may be controlled to different pressures over at least a portion of, or for the entirety of, each initialization test. Each initialization test may have a different controlled pressure at the exhalation valve, and the controlled pressure may be the same or different from the closing pressure described in the pre-initialization test. In an example, the second, third, fourth, fifth, sixth, and seventh initialization tests exhibit controlled pressures at the exhalation valve of stepdown pressures 25 cmH₂O, 20 cmH₂O, 15 cmH₂O, 10 cmH₂O, 5 cmH₂O, and 0 cmH₂O, respectively. Measurement sets may be collected during each of the initialization tests at each flow increment. For example, the ventilator may collect a plurality of first measurement sets for the first initialization test 416 (at the first pressure command for each flow level), a plurality of second measurement sets for the second initialization test 418 (at the second pressure command for each flow level), a plurality of third measurement sets for the third initialization test (at the first stepdown pressure command for each flow level), etc. All or a subset of measurement sets may be used to derive Rohrer's constants in the models described above, for calculating a delivered interface pressure. In between each of the initialization tests, the ventilator may exhibit a transient response, similar to the first transient response and the second transient response, for a time period.

As a further example of the calibration process, the following example of a calibration sequence may be performed by a ventilator:

(1) Connect the ventilator interface to the breathing circuit to conduct a first stage of a pre-initialization test. Apply a relatively high pressure command (e.g., closing pressure) to the exhalation valve to close the valve. For example, the closing pressure commanded to the exhalation valve may be 60 cm H₂O.

(2) While continuing to command the closing pressure on the exhalation valve (e.g., 60 cm H₂O), command the inhalation flow valve to a constant flow (e.g., 40 L/min), and record a first preliminary measurement set including inhalation flow, exhalation flow, inhalation pressure, and exhalation pressure.

(3) Repeat step (2) with a flow stepped down by 2 L/min from the previous flow command for a time interval (e.g., 600 ms), until the measured exhalation flow is negligible (e.g., approximately 0 L/min). At each stepdown, record inhalation flow, exhalation flow, inhalation pressure, and exhalation pressure.

(4) Conduct the second stage of the pre-initialization test. Continue step (3) without maintaining a closing pressure on the exhalation valve, reducing the flow stepdown increment (e.g., to 1 L/min), increasing the consecutive time intervals (e.g., to 1 second), and step down the flow to a baseline flow of 1 L/min. At each step down, record a second preliminary measurement set including inhalation flow, exhalation flow, inhalation pressure, and exhalation pressure.

(5) Conduct a first initialization test. Apply a first pressure command to the exhalation valve (e.g., 30 cmH₂O). Command the inhalation flow valve to a constant flow for a period of time (e.g., 600 ms), and gradually step down the inhalation flow command by 2 L/min, until the measured exhalation flow is less than a negligible threshold value (e.g., 0.3 L/min). At each step down in flow, record a first measurement set including inhalation flow, exhalation flow, inhalation pressure, and exhalation pressure.

(6) Conduct second and subsequent initialization tests. Repeat step (4) at different exhalation valve pressure commands that are held constant throughout the initialization test. At each step down in flow during the test, for the particular exhalation valve pressure command, record measurement sets including inhalation flow, exhalation flow, inhalation pressure, and exhalation pressure. For example in this step, step (4) may be repeated at different stepdown pressure commands such as 25 cmH₂O, 20 cmH₂O, 15 cmH₂O, 10 cmH₂O, 5 cmH₂O, and 0 cmH₂O.

FIG. 4B is a graphical illustration 400B of parameters measured and collected during an example calibration of a non-invasive, non-resistive interface where interface resistance 330 is assumed to be zero (e.g., a mask). As shown, the graphical illustration 400B shows inhalation flow 426, exhalation flow 428, inhalation pressure 430, and exhalation pressure 432 graphed as a function of time as measured by a ventilator. To apply the above calculations to derive an estimated value for the delivered interface pressure, the ventilator runs a calibration routine. The calibration routine depicted in FIG. 4B operates a test 424 to collect a plurality of data points. In this example, there is a single test 424. In other examples, there may be more or less than the single test as shown. More initialization tests may produce more accurate delivered interface pressure calculations but may take more time, while less initialization tests may result in less accurate estimates in a shorter time.

During this example calibration for a non-resistive patient interface (e.g., with a mask interface or other patient interface with minimal interface resistance), the patient circuit and non-invasive interface are connected to the ventilator, but not to the patient. The non-resistive patient interface is blocked to allow gases to, at least in part, exit through the exhalation limb to measure exhalation flow 428 and exhalation pressure 432. During the test 424, the ventilator may fully open the exhalation valve and keep the exhalation valve fully open for the entire test 424. For example, the exhalation valve may be in an open position to maintain approximately atmospheric pressure (or near or slightly above atmospheric pressure) for the entirety of the test (e.g., by applying a 0 mA current to the exhalation valve, in the case of a non-resistive interface). Additionally, during test 424, the ventilator may command a peak calibration flow 434 for a first time interval and reduce the flow command by a flow stepdown increment over a set of consecutive time intervals until the flow command is a desired minimum flow. For example, during the test 424, the peak calibration flow 434 may be 40 L/min for a first time interval of 250 ms, with a flow stepdown increment of 1 L/min, and desired minimum flow of 1 L/min. At each flow increment (e.g., 40 L/min, 39 L/min, 38 L/min, . . . , 1 L/min), the ventilator may record a measurement set including an inhalation flow measurement, inhalation pressure measurement, exhalation flow measurement, and exhalation pressure measurement, which are compiled into a plurality of measurement sets for the test 424.

As a further example of the calibration process, the following example of a calibration sequence may be performed by a ventilator, for a non-resistive interface:

(1) Connect the non-resistive interface to the breathing circuit to conduct a test. Block the non-resistive interface (blocking flow from exiting at the anterior open end of the interface).

(2) Apply a 0 mA current command to the exhalation valve to fully open the exhalation valve.

(3) While continuing to command the exhalation valve to open (e.g., 0 mA current), command the inhalation flow valve to a peak calibration flow (e.g., 40 L/min) for a time interval (e.g., 250 ms), and record a measurement set including inhalation flow, exhalation flow, inhalation pressure, and exhalation pressure.

(4) Repeat step (3) with a flow stepped down by 1 L/min from the previous flow command for a time interval (e.g., 250 ms), until the flow reaches a desired minimum flow (e.g., 1 L/min). At each stepdown, record inhalation flow, exhalation flow, inhalation pressure, and exhalation pressure.

The calibration methods described herein with respect to FIG. 4A and FIG. 4B may be implemented on a ventilator display, such as display 110, which allows performance of this calibration with a selection of a GUI interface button by a user. After the calibration routines are executed, the measurement sets are used to derive Rohrer's constants for the connected interface. The Rohrer's constants are saved as a model of the connected interface, and thereafter the ventilator can use the Rohrer's constants to calculate an anterior interface pressure value during active ventilation of the patient, according to the equations above (derived from FIGS. 3A-3B).

After the ventilator 102 is calibrated, the clinician 108 may set a desired or target anterior interface pressure, or anterior interface pressure range, that may be used by the ventilator 102 in connection with parameter adjustments or alarm triggers. For example, a clinician may set a desired anterior interface pressure and, in response, the ventilator 102 may either display a range of adjustable parameter values or settings for the clinician 108 to set, that correspond with the desired anterior interface pressure, or the ventilator 102 may automatically adjust the flow or pressure commands to target the desired anterior interface pressure. As an additional example, if the anterior interface pressure moves outside of an acceptable range, the ventilator 102 may respond in a variety of ways. For example, the ventilator 102 may cause an alarm to sound, may adjust the flow or pressure command or other settings to cause the anterior interface pressure to reenter the acceptable range, or may display suggested flow, pressure, or other settings that may be commanded by a clinician 108 to cause the anterior interface pressure to fall back within the acceptable range. Additionally or alternatively, the anterior interface pressure may be displayed by the ventilator 102, used in part to control dynamic ventilation, used in part to determine a disconnect or reconnect of the interface, or other uses.

The methods disclosed herein to estimate the interface pressure at the anterior tip can be used for invasive interfaces as well, such as endotracheal tubes. For example, an endotracheal tube can be modeled as an interface with an associated resistance as in FIGS. 3A-B, and the ventilator can then model pressure and flow across the endotracheal tube in the same manner as described above for a nasal interface. In this way, the ventilator can estimate, display, and target the pressure delivered across the anterior end of the endotracheal tube inside a patient's trachea.

In an embodiment, multiple calibrations may be performed for different interfaces, with each interface undergoing a separate calibration routine, which may result in different calibration test data. A patient can then be switched between different, pre-calibrated interfaces without needing to pause ventilation to calibrate.

When one or more different interfaces are calibrated, the associated calibration data (e.g., measured test data, calculated resistance values from the measured test data, etc.) may be used by the ventilator to store a model associated with each interface (for example, Rohrer's constants as described above). The ventilator then shows these calibrated interfaces as available options for selection on a display 110 of the ventilator. For example, a calibration may be performed for a breathing circuit connected to nasal prongs and another calibration may be performed for a breathing circuit connected to a mask. In this example, each set of calibration data is used to create and store respective models on the ventilator for later selection by a clinician when delivering ventilation via a nasal cannula or via a mask, respectively. This may allow for a clinician to switch the patient interface for ventilating a patient without recalibrating the ventilator for the new interface. For example, when ventilating a neonatal patient, nasal prongs may cause trauma to the nares of the patient over time and the clinician may wish to switch to ventilation via a mask. Similarly, the mask may cause irritation or trauma to the face of the neonatal patient over time and the clinician may wish to return to ventilation via a nasal cannula. Indeed, ventilation of neonatal patients may require repeated switching from a nasal cannula to a mask and back to a nasal cannula during treatment. In this case, if both the nasal cannula and the mask have been previously calibrated, recalibration is not necessary, and patient disconnect time is reduced. In further examples, calibrations can be performed for nasal prongs, a nasal or mouth mask, or different sizes of these interfaces, so that a clinician may switch a patient interface on a breathing circuit from prongs to a mask, or from a mask to prongs, or from a mask or prongs of one size to a mask or prongs of a different size. In this case, the clinician may then select an available interface (meaning one that has a stored model) on the display 110 of the ventilator and may continue ventilation without pausing to calibrate the new interface. In this way, switching of the interfaces between stored, calibrated models corresponding to each interface may be performed a plurality of times for a single patient.

Additionally or alternatively, a calibration may be performed after the patient has already begun ventilation. In one example, a clinician may change a breathing circuit after initiating ventilation. In this example, the patient may be disconnected from the breathing circuit while the ventilator runs a calibration test for the changed circuit, reconnecting the patient to the circuit after completion of the calibration test.

Additionally or alternatively, models of various interfaces may be stored on the ventilator from earlier calibration and then used by a user (a medical professional) on a new patient without the user running any additional calibration tests. In an example, models may be determined from calibration data previously obtained by the ventilator (such as calibrations run by the user at an earlier time, or by the manufacturer or service provider). For example, a model of a nasal prong interface that has been previously stored may be used to derive (such as extrapolate) a model for the same type of nasal prongs of a different size. As another example, the ventilator may store an interface model for several possible interfaces connectable to the circuit, which may each have been individually calibrated with a corresponding, stored model.

In an example, the estimated value of the anterior interface pressure can be used by the ventilator to determine that the patient is no longer connected to the interface, or that the interface is no longer connected to the patient (e.g., “patient disconnect”). In some instances, even when disconnected from the patient, a non-invasive interface such as a nasal cannula may have sufficient resistance to cause some gases to flow back through the exhalation limb of the breathing circuit rather than escaping out the interface. This flow of gases through the exhalation limb may cause the ventilator to falsely determine that the non-invasive interface is connected to the patient when it is not. This is because a ventilator may determine patient connection by measuring a threshold flow through the exhalation limb. Additionally, patient disconnection may also be difficult to determine via clinician inspection of the interface (e.g., in the case of non-invasive interfaces), as it may not be readily apparent whether the interface is too loose. By using the estimated anterior nares pressure (delivered interface pressure) when providing ventilation with a nasal cannula, at least as one variable in determining patient disconnect, patient disconnections may be more accurately identified.

In an embodiment, if an estimated anterior nares pressure (delivered interface pressure) crosses below a threshold value, then the ventilator determines that the patient is disconnected from the circuit. For example, the threshold value to determine patient disconnect from the breathing circuit is 0.75 cm H₂O. For example, the ventilator may evaluate the delivered interface pressure over a period of three breaths before determining patient disconnect. As another example, the ventilator may evaluate the delivered interface pressure in control cycle count increments of 5 ms. A determination of patient disconnect from an estimated pressure may additionally be based on an amount of time that the anterior pressure value remains below the threshold, or after a specified number of breaths has elapsed.

Additionally or alternatively, extubation of an endotracheal tube may be determined by using an estimated pressure at the anterior end of the endotracheal tube and a threshold tube pressure. Extubation refers to the removal of the endotracheal tube or dislodging of the endotracheal tube from the patient such that leak is increased and the desired inhalation flow is no longer being delivered to the patient. For example, if an estimated anterior pressure at the end of the endotracheal tube falls below a threshold value, then the ventilator may determine extubation (e.g., that the endotracheal tube is dislodged from the patient).

If the ventilator determines that the ventilator is disconnected from the breathing circuit, the breathing circuit is disconnected from the interface, or the interface is disconnected from the patient, then the ventilator may enter a disconnect phase. In an embodiment, during the disconnect phase, the ventilator monitors for reconnection of the patient. FIG. 4C is a graphical illustration 400C of baseline flow 436 during a disconnect phase to detect patient reconnection to a non-invasive interface. During disconnect phase, the ventilator commands a baseline flow 436 through the inhalation limb and measures a pressure and flow at the exhalation limb to determine if a patient has been reconnected to the circuit. However, a constant baseline flow may not be sufficient to determine reconnect of a patient to a non-invasive interface, as the higher resistance of the non-invasive interface results in higher pressures and flows maintained at the exhalation limb even when the patient is disconnected. In one example, when a non-invasive interface is used, the baseline flow 436 administered during disconnect phase is increased (relative to the baseline flow that is used during disconnect phase with invasive interfaces). For example, a higher baseline flow 436 of 13 L/min may be sufficient to determine reconnect for a non-invasive interface (e.g., higher than 10 L/min). In another example, baseline flow 436 is pulsed to a pulse flow 438 (which is higher than the baseline flow 436) in specified time increments. In an example, the pulse flow 438 is 13 L/min. If these pulses are not detected at the exhalation limb, then the ventilator may determine that a patient has been reconnected. Oscillation between the pulse flow 438 and the baseline flow 436 may be repeated as specified time intervals until patient reconnection is determined. In the example depicted in FIG. 4C, baseline flow 436 may be commanded at 10 L/min for 5 seconds, increased to pulse flow 438 commanded at 13 L/min for a duration of 500 milliseconds, thereafter commanding the baseline flow 436 at 10 L/min for 5.5 seconds followed by pulse flow 438 at 13 L/min as may be repeated until patient reconnect has been detected.

Reconnect based on the baseline flow 436 and/or pulse flow 438 may be determined based in part on the estimated anterior pressure at the interface. For example, the ventilator may determine reconnect when the estimated delivered interface pressure is at least 1.25 cmH₂O for 100 consecutive milliseconds during exhalation phase. Additionally or alternatively, other variables may be used to determine patient reconnect, such as measured exhalation flow which may be compensated to dry flow, inhalation pressure, exhalation pressure, time interval, or other delivered, measured, calculated, or estimated variable, in any combination. For example, patient reconnect may be determined when a threshold anterior nares pressure (delivered interface pressure) (e.g., for a breathing circuit with nasal prongs) is estimated for a specified time interval, when a threshold exhalation flow is measured for a specified time interval, when both inhalation and exhalation pressure are measured to be greater than a threshold pressure for a specified time interval, when inhalation pressure is measured to be greater than a threshold pressure for a specified time interval, a threshold number of control cycle counts, a threshold number of breathing cycles, or any other variable, in any combination.

A calibration method is shown in a flowchart in FIG. 5C. In an example, a method 550 is provided for calibrating a mechanical ventilator for delivered interface pressure monitoring. The method includes displaying, on a user interface of the mechanical ventilator, a prompt to connect an interface (such as a non-invasive interface) to the dual-limb breathing circuit that is connected to the ventilator, at 551. The method includes receiving a user confirmation of connection of the non-invasive interface at 552, and then controlling an inhalation valve to deliver flow to the non-invasive interface at 553. The method includes iteratively reducing the flow by a stepdown increment until the flow reaches a baseline at 554, and collecting a pressure measurement and a flow measurement for each stepdown increment at 555. The method includes storing a model of the non-invasive interface based on the collected pressure and flow measurements at 556. This model may be, for example, a set of Rohrer's constants that characterize the resistance of the patient circuit and connected interface. Once ventilation has resumed, the method includes determining and displaying a delivered interface pressure value based on the stored model at 557.

A method of estimating delivered interface pressure from measured air flow parameters is shown in the flowchart of FIG. 5D, using the example of nasal prongs. A method 570 for controlling a mechanical ventilator includes pausing ventilation to calibrate the ventilator with a non-invasive interface comprising a pair of nasal prongs, at 571. The method includes controlling a flow valve of the ventilator to execute a stepdown flow calibration with the nasal interface, at 572, and updating a circuit model with a plurality of measurements from the flow calibration, at 573. This circuit model may be, for example, a set of Rohrer's constants that characterize the resistance of the patient circuit and connected interface. The method includes resuming ventilation at 574, comprising delivering breathing gases to the nasal interface. After the flow calibration is complete, the ventilator may resume ventilation automatically when it detects connection of a patient to the interface. Then, during active ventilation, the method includes determining a delivered interface pressure value from the updated circuit model, at 575. The interface pressure value comprises a pressure delivered at an anterior end of the nasal prongs. The method includes displaying the delivered interface pressure value on a display screen of the ventilator at 576.

Optionally, the method also includes receiving a target interface pressure (such as from a user) and controlling the ventilator flow valves to drive the delivered interface pressure value toward that target value. A value for Pₙₐᵣₑₛ or Pₘₐₛₖ is set as the pressure target, and the ventilator controls the flow valves to target that set value. With the availability of the pressure estimation at the nares, the settings can be potentially targeted for the pressure at the interface (prongs for NIV, or ETT for invasive). The pressure is regulated by controlling the flow commanded by the inhalation flow valve, and controlling the exhalation valve pressure command (the position and thus degree of opening of the exhalation valve).

The method may also include using the delivered interface pressure value to control patient disconnect alarms, such as the alarm for patient disconnection. In an example, this disconnection (of the patient from the breathing circuit—such as a removal of the interface from the patient, or a disconnection of the interface from the breathing circuit) is detected if the delivered interface pressure value drops below a threshold. The threshold can be a pre-set value stored in the system, or can be adjustable and set by a user. In an example, the threshold is low (such as 0.75 cmH₂O or lower), so that the pressure value dropping below this threshold indicates that the interface or circuit is no longer interacting with the patient's airway. If the interface or circuit becomes disconnected from the patient, the pressure delivered across it encounters less resistance and drops. Thus, the Pₙₐᵣₑₛ or Pₘₐₛₖ value crossing below this threshold can be used to detect this disconnect condition. Without this Pₙₐᵣₑₛ or Pₘₐₛₖ value, the disconnection of the patient from the breathing circuit can be more difficult to identify, leading to nuisance alarms especially in the presence of large or variable leaks during non-invasive ventilation.

In an example, a method includes executing a calibration routine for a non-invasive interface, updating a stored model, resuming active ventilation, obtaining a value of airway pressure (or pressure at the wye) from a proximal flow sensor or from estimations from other sensors, and then determining delivered interface pressure from the airway pressure and the updated model. FIG. 5A is a flow diagram illustrating a method for delivered interface pressure monitoring though pre-ventilation calibration of a ventilator. In an example, method 500 may be performed by a ventilator, such as ventilator 102.

Method 500 begins at operation 502, where the ventilator performs a first initialization test. FIG. 5B is a flow diagram illustrating a method for a ventilator to carry out the first initialization test of operation 502. An initialization test may have several steps to obtain sets of data measurements at the inhalation and exhalation ports of the ventilator. For example, initialization tests may be performed by varying a flow and/or a pressure command, while measuring inhalation flow, inhalation pressure, exhalation flow, and exhalation pressure, to collect sets of inhalation and exhalation data.

For example, FIG. 5B depicts an example of a first initialization test. In this example, the test is performed by controlling an exhalation valve to maintain a first pressure (such as 30 cmH₂O) over a set of first consecutive time intervals, at operation 502a; commanding a flow (such as 40 L/min) for a time interval (such as 600 ms) of the first set of consecutive time intervals, at operation 502b; reducing the flow by a predefined flow stepdown increment (such as 2 L/min) for each of the first set of consecutive time intervals until the flow command reaches a baseline flow (such as 10 L/min), at operation 502c; and collecting a plurality of first measurement sets, wherein each of the plurality of first measurement sets corresponds to a flow level and includes an inhalation flow measurement, an inhalation pressure measurement, an exhalation flow measurement, and an exhalation pressure measurement, at operation 502d. These measurements are compiled into a measurement set. Operations 502a-d may be further described herein with respect to FIG. 4A (for resistive interfaces). Alternatively, operations 502a-d may perform a single test (e.g., for a non-resistive interface or a mask), as further described herein with respect to FIG. 4B.

In some examples, operation 502 may be repeated for a variety of different pressure commands (e.g., for each set of flow stepdown operations). The different pressure commands may include, for instance, 30 cmH₂O, 25 cmH₂O, 20 cmH₂O, 15 cmH₂O, 10 cmH₂O, 5 cmH₂O, 0 cmH₂O. In this case, for each initialization test, the pressure may be stepped down by a pressure stepdown increment (e.g., 5 cmH₂O). The collected measurement set from each flow stepdown operation at the set pressure command may then be compiled as a plurality of measurement sets.

Returning to method 500 in FIG. 5A, after performing operation 502 (as illustrated by FIG. 5B) and any repeated pressure command varieties of operation 502, operation 504 may be performed. At operation 504, the plurality of first measurement sets may be used to determine an inhalation resistance 326 and an exhalation resistance 328 by determining inhalation Rohrer's constants and exhalation Rohrer's constants, respectively. The Rohrer's constants may be determined by fitting or modeling the first measurement sets. For example, the first measurement sets may be fitted or modeled using least squares estimations, averages, or other fitting methods or models. Measured interval data sets from operation 502, in addition to any interval data sets collected from repeats of operation 502, may be used to determine a nonlinear relationship between the inhalation resistance and inhalation flow, and exhalation resistance and inhalation flow, as described above such as by using least square method.

At operation 506, a prong subset of the plurality of the first interval measurement sets may be used to determine a prong resistance by calculating prong Rohrer's constants. This prong subset may be associated with portions of interval data sets having a negligible exhalation flow. When the exhalation flow is negligible, the net flow through the prong will be the same as the inhalation flow because the exhalation flow is essentially zero, and the pressure gradient across the prong will be the same as the exhalation pressure because the pressure gradient is zero from the prong wye junction to the exhalation valve, due to zero exhalation flow. Using the prong subset, a similar process for determining inhalation and exhalation resistance is used to determine the prong resistance, as described above. The negligible exhalation flow may be associated with the baseline flow, to ensure that at least some measurement sets are collected when the exhalation flow is assumed to be negligible. A “negligible” exhalation flow value may be set by a clinician. For example, a negligible exhalation flow may be a flow less than 0.3 L/min.

After determining inhalation resistance, exhalation resistance, and prong resistance as a function of commanded flow, the method proceeds to operation 508 where the ventilator may estimate or measure an airway pressure. One way to do this is by obtaining a pressure measurement from a sensor at the wye. Another way is to estimate the airway pressure from sensors at the ventilator. In an example, airway pressure is estimated by calculating an inhalation airway pressure and an exhalation airway pressure and then combining those two values (such as by minimizing a variance between them). The inhalation airway pressure may be calculated by using the calculated inhalation resistance, measured inhalation pressure and measured inhalation flow. A similar process may be used to calculate the exhalation airway pressure, as shown in the following equations:

P_{aw,i}=Pᵢ−Rᵢ·Qᵢ=Pᵢ−(β_{0,i}+β_{1,i}·Qᵢ)·Qᵢ

P_{aw,i}=Pₑ−Rₑ·Qₑ=Pₑ−(β_{0,i}+β_{1,i}·Qₑ)·Qₑ

The calculated airway pressure P_{aw} may then be used along with the prong resistance to calculate a delivered interface pressure, at operation 510. For example, as described above, the delivered interface pressure may be calculated by subtracting the product of the prong resistance and the net flow (inhalation flow minus exhalation flow) from the airway pressure:

ΔP_{prong}=R_{prong}·Qₙₑₜ=(β_{0,prong}+β_{1,prong}·Qₙₑₜ)·Qₙₑₜ

βₙₐᵣₑₛ=P_{aw}−ΔP_{prong} At operation 512, the ventilator may display the current delivered interface pressure. Additionally or alternatively, the ventilator may automatically control the inhalation and/or exhalation valve (commanding a certain flow or pressure) to reach a target delivered interface pressure. This target delivered interface pressure may be set by a clinician, such as clinician 108, or by data available to the ventilator about suitable delivered interface pressure for a given patient.

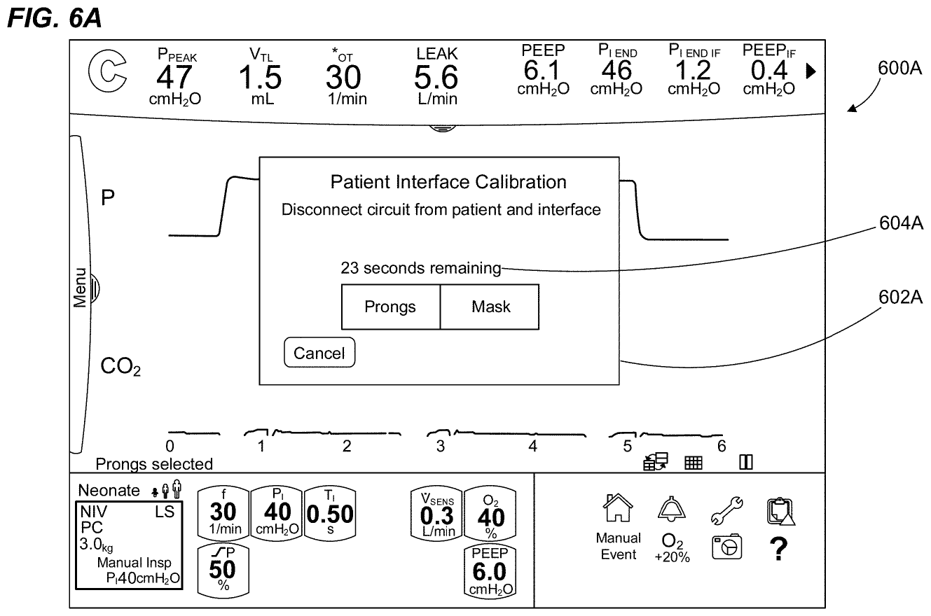

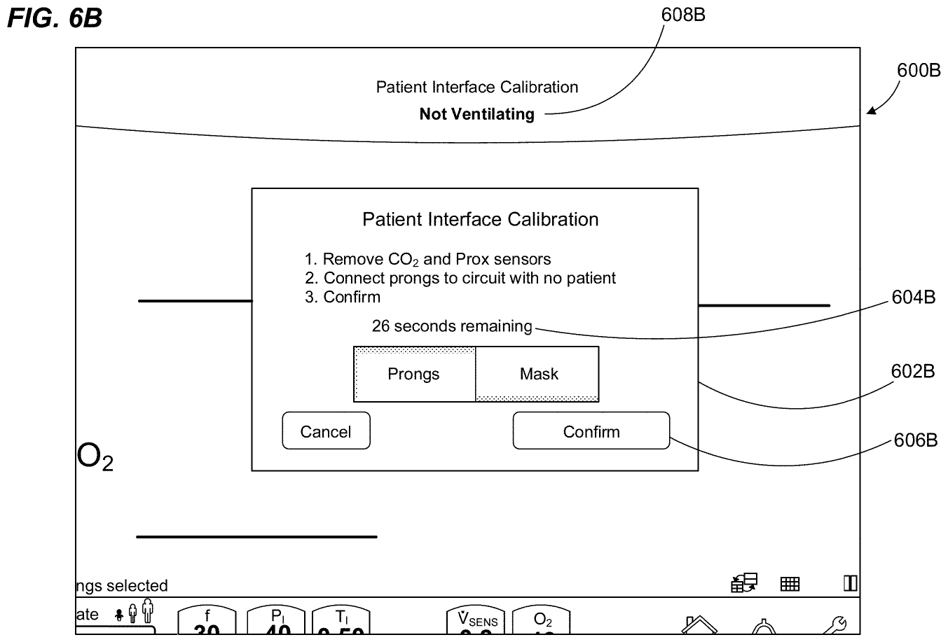

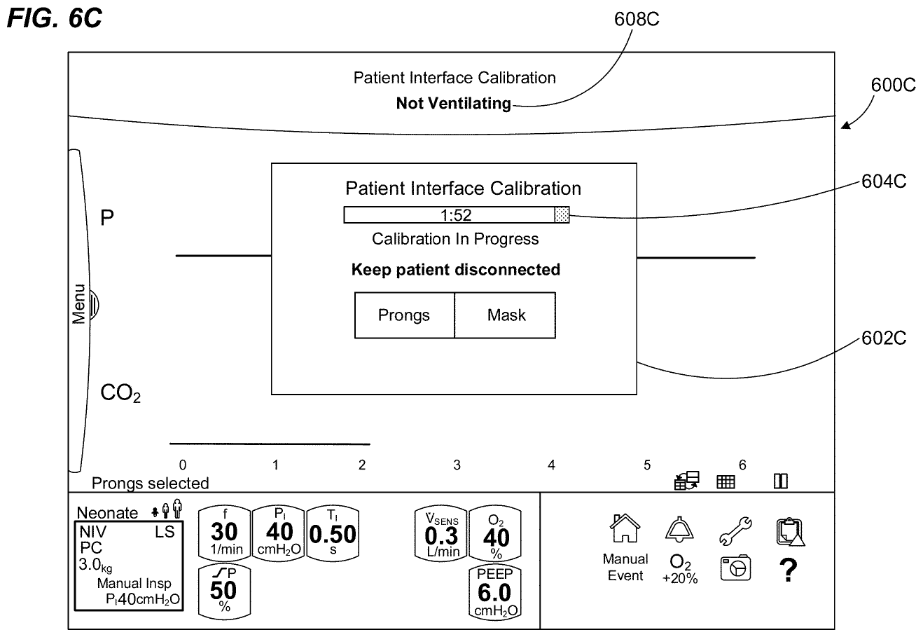

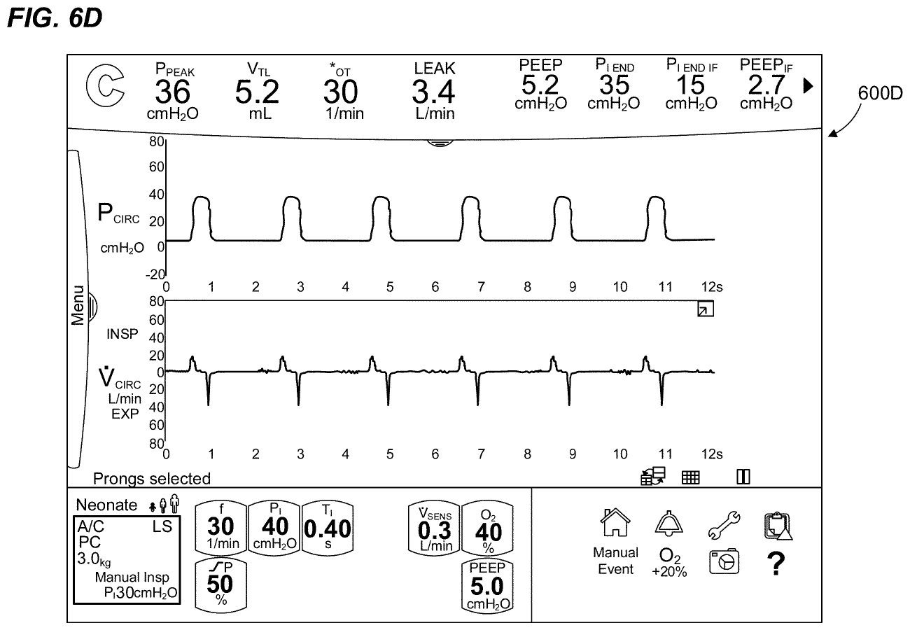

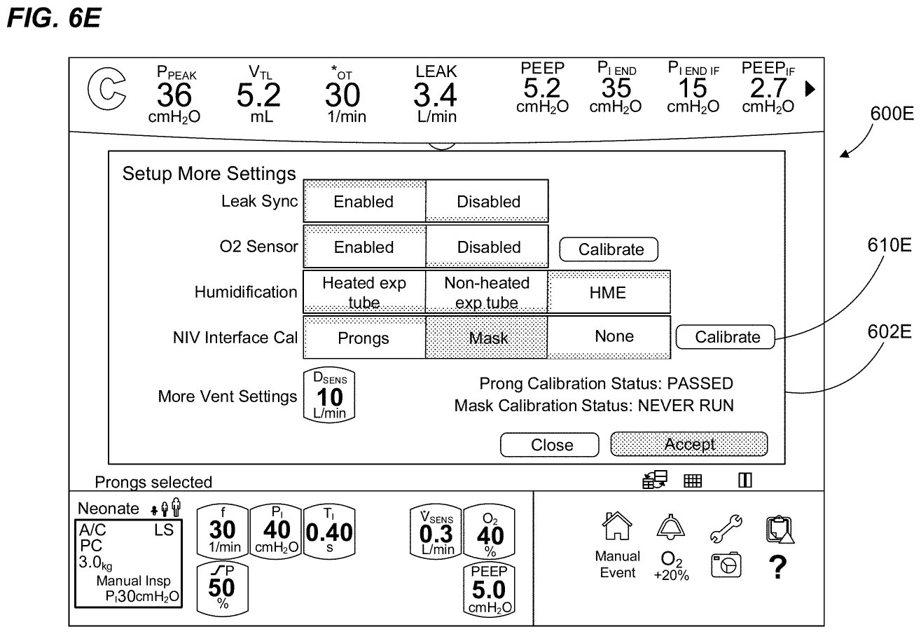

FIGS. 6A-E are representations of a graphical user interface for delivered interface calibration and pressure monitoring, according to an example. In an example, the graphical user interface (GUI) includes inputs for a user to select non-invasive ventilation and then request to calibrate a non-invasive interface such as nasal prongs or a mask. The GUI workflow includes prompts and screens that walk the user through the calibration process to prepare the ventilator to begin non-invasive ventilation with delivered interface pressure monitoring or control.

In an example, the GUI includes a window or screen where a user selects non-invasive ventilation, and then requests a new calibration. The ventilator then prompts the user to disconnect the circuit from the patient. An example ventilator screen 600A is shown in FIG. 6A. In this figure, the ventilator screen 600A displays a prompt window 602A with a message that prompts the user to disconnect the circuit from the patient (and from the patient's previous interface, such as a previous mask, nasal prongs, endotracheal tube, or other interface). While this prompt window 602A is displayed, the ventilator is waiting to automatically detect the disconnection of the patient from the circuit. In an example, the ventilator also sets a countdown timer 604A (such as 30 seconds), and if the countdown timer 604A ends before disconnection is detected, the ventilator will remove the prompt window 602A and return to the previous ventilator screen 600A.

If the ventilator detects the disconnection of the dual-limb circuit from the patient, the ventilator displays the ventilator screen 600B shown in FIG. 6B. In this ventilator screen 600B, the ventilator displays a prompt window 602B with a message that prompts the user to select a type of non-invasive interface (such as nasal prongs or mask) and connect that interface to the dual-limb circuit. If the prongs are selected and confirmed, the user will be prompted to leave the anterior end of the interface open to ambient air (not attached to the patient). (If Mask is selected and confirmed, the user will be prompted to block the mask during calibration.) The prompt window 602B in FIG. 6B also provides instructions to the user to remove downstream sensors (downstream of the ventilator, such as CO₂ or proximal flow sensors). The prompt window 602B also prompts the user to confirm that the interface has been so connected. Again, a countdown timer 604B may run at the same time, and the ventilator exits the prompt window 602B if it does not receive a “Confirm” input 606B before the countdown timer 604B expires.

While the ventilator screen 600B in FIG. 6B is displayed, the patient has already been disconnected from the breathing circuit, and thus the ventilator is not actively ventilating a patient. As described above, this may be performed prior to any patient connection to the ventilator, or may be performed when a patient is disconnected from the ventilator. The GUI (e.g., ventilator screen 600B) includes a corresponding “not ventilating” message 608B (or alert, etc). In an example, the “not ventilating” message 608B also displays an amount of time that has passed during which the ventilator has been in this state where it is not ventilating a patient.

If the ventilator receives the “Confirm” input 606B, it begins a calibration routine to calibrate the connected non-invasive interface. During the calibration routine, the ventilator displays a progress screen 602C (or status screen), such as shown in FIG. 6C. This progress screen 602C displays a progress bar or other status information to the user during the calibration, such as to show the amount of time that has passed during the calibration and/or the amount of time remaining. In an example, this progress screen 602C displays a countdown timer 604C that counts down from the total time duration of the calibration routine, such as two minutes (or 30, 60, or 90 seconds, or other suitable duration). As in FIG. 6B, during this time, the ventilator is not actively ventilating a patient, and the GUI (e.g., ventilator screen 600C) includes a corresponding “not ventilating” message 608C (or alert) on the screen. In an example, the “not ventilating” message 608C also displays an amount of time that has passed during which the ventilator has been in this state where it is not ventilating a patient.

If the calibration is unsuccessful, the ventilator displays a message or alert to the user with associated information. If the calibration is successful, the ventilator displays another screen or window that prompts the user to connect the calibrated interface to the patient. In an example, as soon as the interface is connected to the patient, the ventilator automatically detects the connection and begins ventilation of the patient. The ventilator stores a model of the calibrated interface, and uses that model to determine anterior interface pressure during ventilation. As discussed above, multiple calibrations can be run for different interfaces, and the ventilator can store a different model for each different calibrated interface (such as masks of various sizes and/or nasal prongs of various sizes). The user can switch the patient to any of these pre-calibrated interfaces and then select that interface on the ventilator, during ventilation without requiring an additional calibration after initiating ventilation.

FIG. 6D shows a GUI screen (e.g., ventilator screen 600D) during active ventilation of a patient via a non-invasive interface, with delivered interface pressure displayed on the ventilator screen 600D. The pressure and flow waveforms show active ventilation (breaths delivered to the patient), and two delivered interface pressure values are displayed at the top of the screen. The P_{I END IF} value shows the anterior interface pressure at the end of inspiration, at the anterior tip of the nasal interface. The PEEP_{IF} value shows the anterior interface pressure at the end of expiration, at the anterior tip of the interface. (“IF” stands for the interface.) The target values for inhalation pressure (P_{I}) and PEEP are shown in the bottom panel on the screen, where the target P_{I} is set to 40 cmH₂O and the target PEEP is set to 5.0 cmH₂O. The pressure values at the airway—not at the anterior end of the nasal interface, but upstream at the wye—are shown at the top of the screen as P_{I END} (shown as 35 cmH₂O) and PEEP (shown as 5.2 cmH₂O). However, the pressure actually delivered across the interface is much lower than that. In this example, P_{I END IF} is 15 cmH₂O and PEEP_{IF} is 2.7 cmH₂O. The difference in values (from 35 to 15, and from 5.2 to 2.7) shows the drop in pressure across the patient interface, before the breathing gases are actually delivered to the patient.

FIG. 6E shows a GUI screen (e.g., ventilator screen 600E) during active ventilation of a patient via a non-invasive interface, with active ventilation settings displayed on the ventilator screen 600E. In this ventilator screen 600E, the ventilator displays a settings window 602E with currently selected inputs for the ongoing ventilation. For example, the settings window 602E may include an “Enabled” input and “Disabled” input associated with Leak Sync and/or O2 Sensor. As another example, Humidification may be associated with the following inputs: “Heated exp tube” input, “Non-heated exp tube” input, and/or “HME” input. Additionally the settings window 602E may include More Vent Settings, such as a flow to be commanded after detecting patient disconnect (D_{SENS} 10 L/min). As another example, the settings window 602E may include inputs associated with non-invasive interface calibrations (“NIV Interface Cal”). For example, NIV Interface Cal may include a “Prongs” input if prongs have been previously calibrated and/or a “Mask” input if a mask has been previously calibrated. Input options may not be available for selection if an input is not associated with a stored model associated with calibration data. Additional interfaces may be added as input options by selecting the “Calibrate” input 610E. A selection of the “Calibrate” input 610E may prompt calibration GUIs for additional input, such as the prompt windows (e.g., prompt windows 602A-D and settings window 602E) and ventilator screens (e.g., ventilator screens 600A-E) described herein with respect to FIGS. 6A-6C. In the example shown in FIG. 6E, the “Mask” input is non-selectable because there was no previously stored model for the mask at this ventilator. This may be further indicated by a message in the settings window 602E: “Mask Calibration Status: NEVER RUN.” If the “Calibrate” input 610E is selected and a calibration is run for a mask (e.g., as described in FIGS. 6A-6C), then reloading of this settings window 602E may have the “Mask” input as a selectable option associated with NIV Interface Cal and the message in the settings window 602E may read: “Mask Calibration Status: PASSED.”

Additionally or alternatively, or in combination, the settings window 602E may have a drop-down menu or other type of menu to select inputs associated with NIV Interface Cal. For example, the “Prong” input may have a drop-down menu of selectable prong sizes. As described herein, each prong size may be individually calibrated and an associated model may be stored, or a model may be calculated from one or more prong models based on a predetermined relationship between the different prong sizes. Selectable prong sizes may not require additional calibration.

When an input on the settings window 602E is changed from the previously selected settings, then an “Accept” input may need to be received prior to changing the settings. In this example, if any of the current settings in the settings window 602E are changed, then the “Accept” input may become selectable and may require selection prior to the settings being saved and/or implemented by the ventilator. The settings window 602E may additionally or alternatively be closed, with or without accepting new settings, with a selection of the “Close” input.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

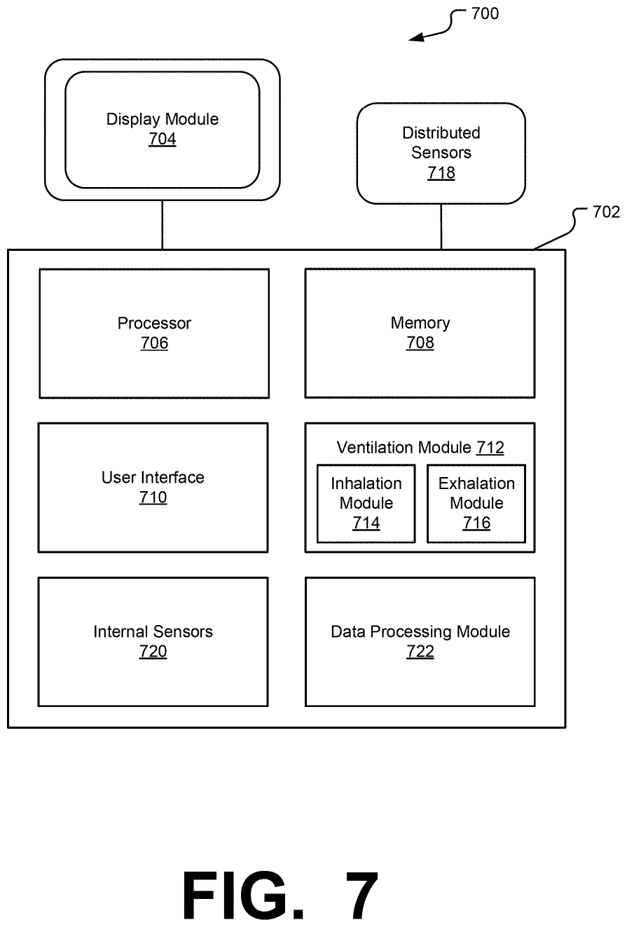

FIG. 7 is a block-diagram illustrating an example of a ventilatory system 700 for clinician decision support. Ventilatory system 700 includes ventilator 702 with its various modules and components. That is, ventilator 702 may further include, inter alia, memory 708, one or more processors 706, user interface 710, and ventilation module 712 (which may further include an inspiration module 714 and an exhalation module 716). Memory 708 is defined as described above for memory 712. Similarly, the one or more processors 706 are defined as described above for one or more processors 716. Processors 706 may further be configured with a clock whereby elapsed time may be monitored by the system 700.

The ventilatory system 700 may also include a display module 704 communicatively coupled to ventilator 702. Display module 704 provides various input screens, for receiving clinician input, and various display screens, for presenting useful information to the clinician. The display module 704 is configured to communicate with user interface 710 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, etc.). The elements may include controls, graphics, charts, tool bars, input fields, icons, etc. Alternatively, other suitable means of communication with the ventilator 702 may be provided, for instance by a wheel, keyboard, mouse, or other suitable interactive device. Thus, user interface 710 may accept commands and input through display module 704. Display module 704 may also provide useful information in the form of various ventilatory data regarding the physical condition of a patient and/or a prescribed respiratory treatment. The useful information may be derived by the ventilator 702, based on data collected by a data processing module 722, and the useful information may be displayed to the clinician in the form of graphs, wave representations (e.g., a waveform), pie graphs, or other suitable forms of graphic display. For example, the data processing module 722 may be operative to monitor delivered interface pressure and may display useful information regarding the delivered interface pressure, as detailed above.

Ventilation module 712 may oversee ventilation of a patient according to ventilatory settings. Ventilatory settings may include any appropriate input for configuring the ventilator to deliver breathable gases to a particular patient, including settings for delivering either mandatory or spontaneous breaths to the patient. Ventilatory settings may be entered by a clinician, e.g., based on a prescribed treatment protocol for the particular patient, or automatically generated by the ventilator, e.g., based on attributes (i.e., age, diagnosis, ideal body weight, gender, etc.) of the particular patient according to any appropriate standard protocol or otherwise. In some cases, certain ventilatory settings may be adjusted to optimize the prescribed treatment, e.g., decreasing a mandatory rate so there is more time for a spontaneous rate in an assist/control-type mode. Other ventilatory settings may include, inter alia, tidal volume (VT), respiratory rate (RR), inhalation time (Ti), inhalation pressure (P_{I}), pressure support (P_{SUPP}), rise time percent (rise time %), exhalation (or exhalation) time (T_{E}), peak flow, flow pattern, etc.

Ventilation module 712 may further include an inspiration module 714 configured to deliver gases to the patient according to prescribed ventilatory settings. Specifically, inspiration module 714 may correspond to the inhalation module 604 or may be otherwise coupled to source(s) of pressurized gases (e.g., air, oxygen, and/or helium), and may deliver gases to the patient. Inspiration module 714 may be configured to provide ventilation according to various ventilatory types and modes, e.g., via volume-targeted, pressure-targeted, or via any other suitable type of ventilation.

The memory (612, 708) includes non-transitory, computer-readable storage media that stores software that is executed by the processor (616, 706) and which controls the operation of the ventilator. In an example, the memory includes one or more solid-state storage devices such as flash memory chips. In an alternative example, the memory may be mass storage connected to the processor through a mass storage controller (not shown) and a communications bus (not shown). Although the description of computer-readable media contained herein refers to a solid-state storage, it should be appreciated by those skilled in the art that computer-readable storage media can be any available media that can be accessed by the processor. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

A ventilator programmed to calibrate a non-invasive interface and estimate a value of Pₙₐᵣₑₛ or Pₘₐₛₖ during active ventilation was tested with two interfaces, as shown below. The interfaces were connected to a test fixture to enable comparison of the ventilator's estimated value of Pₙₐᵣₑₛ or Pₘₐₛₖ to the actual measured pressure at the interface. The results below show good agreement between the ventilator's estimation and the measured values.

TABLE 1

| | | | | | End of | | |
|---|---|---|---|---|---|---|---|
| | | | End of | | Exhalation Pressure @ | | |
| | | | Inspiration Pressure @ | | Interface, Comparison | | |
| | | | Interface, Comparison | | Between Estimated | | |
| | Ventilator Pressure | | Between Estimated | | and Measured (cmH2O) | | |
| | Setting (cmH2O) | | and Measured (cmH2O) | | Estimated | | |
| | Inhalation | | Estimated | | interface | | |
| | Pressure | | interface | Actual | pressure | Actual | Leak Rate |
| Interface | (Above PEEP) | PEEP | pressure | Measured | (PEEP) | Measured | (L/min) |
| Fisher & | 15 | 3 | 9.8 | 9.50 | 2.0 | 2.08 | 2.1 |
| Pavkel ® | 15 | 6 | 12 | 11.05 | 3.9 | 3.73 | 3.2 |
| nasal prong | 15 | 12 | 15 | 14.26 | 7.5 | 7.10 | 5.0 |
| 3020 | 25 | 6 | 17 | 15.86 | 3.9 | 4.02 | 3 2 |
| | 40 | 6 | 24 | 22.64 | 3.9 | 3.87 | 3.2 |
| Neotech ® | 15 | 3 | 15 | 15.41 | 2.6 | 2.59 | 1.0 |
| Ram Cannula | 15 | 6 | 18 | 18.07 | 5.2 | 5.22 | 1.6 |
| N4902 | 15 | 12 | 23 | 23.28 | 10 | 10.33 | 2.7 |
| | 25 | 6 | 27 | 26.83 | 5.6 | 5.17 | 1.6 |
| | 40 | 6 | 40 | 40.11 | 5.1 | 5.23 | 1.6 |

Those skilled in the art will recognize that the methods and systems of the present disclosure may be implemented in many manners and as such are not to be limited by the foregoing exemplary aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for calibrating a ventilator for ventilation via a non-invasive interface, comprising:
displaying, on a user interface of the ventilator, a prompt to connect the non-invasive interface to the ventilator;
receiving a user confirmation of connection of the non-invasive interface;
commanding a flow to the non-invasive interface;
iteratively reducing the commanded flow by a stepdown increment until the commanded flow reaches a baseline;
collecting a pressure measurement and a flow measurement for each stepdown increment;
determining a resistance constant for the interface based on the collected measurements;
storing the resistance constant; and
determining, based on the stored resistance constant, a delivered interface pressure during ventilation; and
performing at least one of the following:
displaying the delivered interface pressure;
controlling the ventilation based at least in part on the delivered interface pressure; or
determining a disconnect or reconnect status based on the delivered interface pressure.

2. The method of claim 1, wherein the resistance constant comprises a Rohrer's constant.

3. The method of claim 1, wherein each stepdown increment is 1 L/min.

4. The method of claim 1, wherein determining the delivered interface pressure comprises measuring or estimating an airway pressure using the resistance constant.

5. The method of claim 4, wherein the airway pressure is estimated from an inhalation airway pressure measured by the ventilator and an exhalation airway pressure measured by the ventilator.

6. The method of claim 4, wherein the airway pressure is measured by a proximal sensor located downstream of the ventilator.

7. The method of claim 1, further comprising adjusting the commanded flow or a controlled pressure of an exhalation valve to target a desired delivered interface pressure.

8. The method of claim 1, wherein the non-invasive interface comprises a nasal cannula comprising nasal prongs, and wherein the delivered interface pressure comprises a pressure delivered across an anterior tip of the nasal prongs.

9. The method of claim 1, wherein the non-invasive interface comprises a mask, and wherein the collecting operation occurs when the mask is blocked, and further wherein the delivered interface pressure comprises a pressure delivered at the mask.

10. The method of claim 1, further comprising triggering an alarm if the delivered interface pressure crosses a threshold.

11. The method of claim 1, wherein the method operates devoid of input from sensors downstream of the ventilator.

12. A method for calibrating a mechanical ventilator, comprising:
displaying, on a user interface of the mechanical ventilator, a prompt to connect a non-invasive interface to a dual-limb patient circuit;
receiving a user confirmation of connection of the non-invasive interface to the dual-limb patient circuit;
commanding a flow to the non-invasive interface via the dual-limb patient circuit;
iteratively reducing the commanded flow by a stepdown increment until the flow reaches a baseline;
collecting a pressure measurement and a flow measurement for each stepdown increment;
determining a set of resistance constants for the non-invasive interface and the dual-limb patient circuit, based on the collected pressure and flow measurements;
determining, based on the set of resistance constants, a delivered interface pressure;
detecting a reconnection of the non-invasive interface to a patient;
automatically resuming ventilation; and
displaying, on the user interface, the delivered interface pressure during ventilation.

13. The method of claim 12, further comprising:
displaying, on the user interface, an option to calibrate one of nasal prongs or a mask, wherein the non-invasive interface corresponds to one of the nasal prongs or the mask; and
receiving a user selection of one of the nasal prongs or the mask.

14. The method of claim 12, further comprising:
displaying, on the user interface, a third prompt to remove at least one of a carbon dioxide (CO₂) sensor or a proximal flow (Prox) sensor from the dual-limb patient circuit.

15. The method of claim 12, wherein the reconnection of the non-invasive interface to the patient is determined based on the delivered interface pressure.

16. The method of claim 12, the method further comprising:
displaying, on a user interface of the mechanical ventilator, a prompt to disconnect the non-invasive interface from the patient; and
receiving a user confirmation of disconnection of the non-invasive interface from the patient.

17. The method of claim 12, wherein the operation of detecting a reconnection of the non-invasive interface to the patient further comprises oscillating between a baseline flow and a pulse flow.

18. A method for controlling a ventilator, comprising:
pausing ventilation to calibrate the ventilator with a nasal prong interface comprising nasal prongs;
controlling a flow valve of the ventilator to execute a stepdown flow calibration with the nasal prong interface open to ambient air;
updating a set of resistance values based on a plurality of measurements from the stepdown flow calibration;
resuming ventilation, comprising delivering breathing gases to the nasal prong interface;
determining a delivered interface pressure from the updated set of resistance values, wherein the delivered interface pressure comprises a pressure delivered at an anterior end of the nasal prongs; and
displaying the delivered interface pressure on a display screen of the ventilator.

19. The method of claim 18, further comprising receiving a target interface pressure and controlling the flow valve to drive the delivered interface pressure toward the target interface pressure.

20. The method of claim 18, further comprising determining a disconnect of the nasal prong interface based on the delivered interface pressure and a threshold pressure.
